(19) United States
(12) Patent Application Publication
(10) Pub. No.: US 2017/0303859 A1
(43) Pub. Date: Oct. 26, 2017

(54) SYSTEMS, DEVICES, AND METHODS FOR TRACKING AND COMPENSATING FOR PATIENT MOTION DURING A MEDICAL IMAGING SCAN

(71) Applicants: KinetiCor, Inc., Honolulu, HI (US); The University of Hawaii, Honolulu, HI (US)

(72) Inventors: Trent Victor Robertson, Honolulu, HI (US); Thomas Michael Ernst, Honolulu, HI (US); William Herman Alameida, JR., Honolulu, HI (US); Aditya Singh, Honolulu, HI (US)

(21) Appl. No.: 15/495,771

(22) Filed: Apr. 24, 2017

Related U.S. Application Data
(60) Provisional application No. 62/327,884, filed on Apr. 26, 2016.

(51) Int. Cl. A61B 5/00; A61B 90/00; A61B 5/11; A61N 5/10

(52) U.S. Cl. CPC A61B 5/721; A61B 5/1127; A61B 90/39; A61B 5/6819; A61N 5/1049; A61B 2090/374; A61B 2090/3904; A61B 2090/3937; A61N 2005/1059; A61N 2005/1087

(57) ABSTRACT

An optical marker apparatus or device for tracking and compensating for patient motion during a medical imaging scan can comprise one or more optical markers, one or mounts, and a substrate. The one or more optical markers can be adapted to be detected by one or more detectors and/or cameras of a medical imaging scanner and/or motion tracking system. One or more mounts can be configured to attach the one or more markers to the substrate. The substrate, in turn, can be configured to be attached to one or more regions of a subject patient's body. For example, in some embodiments, a substrate can be adapted to be placed or otherwise attached over a nose bridge of a patient.

### CROSS-REFERENCE TO RELATED APPLICATIONS

The present application claims the benefit under 35 U.S.C. §119(c) of U.S. Provisional Application No. 62/327,884, filed Apr. 26, 2016, and entitled “SYSTEMS, DEVICES, AND METHODS FOR TRACKING AND COMPENSATING FOR PATIENT MOTION DURING A MEDICAL IMAGING SCAN,” which is hereby incorporated herein by reference in its entirety under 37 C.F.R. §1.57.

### BACKGROUND

The disclosure relates generally to the field of motion tracking, and more specifically to systems, devices, and methods for tracking and compensating for patient motion during a medical imaging scan.

There are various modalities for performing medical imaging of patients. For example, magnetic resonance imaging (MRI) is a medical imaging technique used in radiology to visualize internal structures of the body in detail. An MRI scanner is a device in which the patient or a portion of the patient's body is positioned within a powerful magnet where a magnetic field is used to align the magnetization of some atomic nuclei (usually hydrogen nuclei-protons) and radio frequency magnetic fields are applied to systematically alter the alignment of this magnetization. This causes the nuclei to produce a rotating magnetic field detectable by the scanner and this information is recorded to construct an image of the scanned region of the body. These scans typically take several minutes (up to about 40 minutes in some scanners) and in some devices any significant movement can ruin the images and require the scan to be repeated.

United States Patent Application Publication No. US 2016/0035108, filed on Jul. 22, 2015 and published on Feb. 4, 2016, describes systems, devices, and methods that adaptively compensate for subject motion which may be utilized in combination with and/or in addition to one or more embodiments disclosed herein. United States Patent Application Publication No. US 2016/0035108 is incorporated herein by reference in its entirety and forms part of this specification.

Additionally, there are various radiation therapies, proton therapies, and other therapies that can be applied to patients. For example, radiation therapy can be applied to a targeted tissue region. In some systems, radiation therapy can be dynamically applied in response to patient movements. However, in many such systems, the tracking of patient movements does not have a high degree of accuracy. Accordingly, the use of such systems can result in the application of radiation therapy to non-targeted tissue regions, thereby unintentionally harming healthy tissue while intentionally affecting diseased tissue. The foregoing is also true for proton therapies and other therapies.

### SUMMARY

An accurate and reliable method of determining the dynamic position and orientation of a patient's head or other body portion during MRI scanning or therapeutic procedures is a requirement in any attempt to compensate for subject motion during such procedures. Toward this end, disclosed herein are systems and methods that include practical optical head tracking capability using at least an optical marker apparatus or device, such as a compact, inexpensive target which mounts comfortably and rigidly to the subject's skeletal frame.

In some embodiments, for example, an optical marker apparatus or device 100 comprises one or more optical markers and/or a base portion. The base portion can further comprise a substrate and one or more mounts attached to the substrate. In certain embodiments, the one or more optical markers can be adapted to be detected by one or more detectors and/or cameras of a medical imaging scanner or therapeutic device and/or motion tracking system thereof. In certain embodiments, the base portion and/or substrate thereof can be adapted to be attached to one or more regions of a subject patient's body. For example, in some embodiments, the base portion and/or substrate thereof can be adapted to be placed or otherwise attached over a nose bridge of a patient. In some embodiments, the substrate can be adapted to be directly attached to a subject patient. In certain embodiments, one or more mounts can be adapted to connect the substrate with one or more optical markers.

In some embodiments, an optical marker apparatus for tracking motion of a subject during a medical imaging scan comprises: a substrate configured to be attached on a nose bridge of the subject of the medical imaging scan, the substrate comprising a flexible material configured to be shaped to substantially cover a curvature of the nose bridge of the subject to at least partially contact the nose bridge of the subject, the substrate comprising a longitudinal axis; a first mount connected to a surface of the substrate, wherein the first mount is configured to be placed over a first side of a nose of the subject, wherein the first mount comprises a rigid material; a second mount connected to the surface of the substrate, wherein the second mount is configured to be placed over a second side of the nose of the subject, wherein the second mount comprises a rigid material, wherein the first mount and the second mount are located along the longitudinal axis of the substrate, wherein each of the first mount and the second mount comprises a top surface, wherein the top surface is angled such that it is not parallel to the first side of the nose and the second side of the nose, wherein the top surface comprises one or more patterns, wherein the one or more patterns forms an indicator; a first optical marker and a second optical marker, the first optical marker configured to be attached to the top surface of the first mount, and the second optical marker configured to be attached to the top surface of the second mount, wherein at least one or more of the first optical marker and the second optical marker are configured to be identified by one or more optical detectors for tracking motion of the subject during the medical imaging scan.

In certain embodiments, the optical marker apparatus further comprises a third mount, wherein the third mount is connected to the surface of the substrate, and a third optical marker, wherein the third optical marker is configured to be attached to a top surface of the third mount. In certain embodiments, the third optical marker is configured to be placed substantially above the nose bridge of the subject. In certain embodiments, only a central portion of the third mount is connected to the substrate. In certain embodiments, the third mount comprises a top surface, wherein the top surface of the third mount is substantially parallel to the substrate before attaching to the subject.

In some embodiments, the optical marker apparatus further comprises a third mount, wherein the third mount is connected to the surface of the substrate, wherein the third mount comprises one or more pillars, wherein a third optical marker is configured to be attached to the one or more pillars. In certain embodiments, a central portion of the substrate comprises a void space. In certain embodiments, the void space is configured to be formed after the substrate is attached to the nose bridge of the subject by removing some or all of the central portion.

In some embodiments, a central portion of the substrate comprises a removable portion configured to reduce a thickness of the central portion upon removal of the removable portion. In some embodiments, a central portion of the substrate is thinner than a terminal portion of the substrate. In some embodiments, the substrate is configured to prevent substantial movement of the second mount by an isolated movement of the first mount. In some embodiments, the top surface of the first mount is angled such that the first optical marker is orthogonal to a line of sight of the optical detector when the first optical marker is attached to the first mount. In some embodiments, the top surface of the first mount comprises a recessed area for affixing the first optical marker. In some embodiments, the top surface of the first mount comprises a recessed portion and the first optical marker comprises a protruding portion, wherein the protruding portion of the first optical marker is configured to be inserted into the recessed portion to attach the first optical marker to the first mount. In some embodiments, the indicator is configured to provide a visual guide to a user for attaching the first optical marker to the first mount and for attaching the second optical marker to the second mount. In some embodiments, the first optical marker is configured to be attached to the first mount by a chemical adhesive.

In some embodiments, an optical marker apparatus for tracking motion of a subject during a medical imaging scan comprises: a substrate configured to be attached on a nose bridge of the subject of the medical imaging scan, the substrate comprising a flexible material configured to be shaped to substantially cover a curvature of the nose bridge of the subject to at least partially contact the nose bridge of the subject, the substrate comprising a longitudinal axis; a first mount connected to a surface of the substrate, wherein the first mount is located at a terminal end of the substrate, wherein the first mount comprises a rigid material; a second mount connected to the surface of the substrate, wherein the second mount is located at a central portion of the substrate, wherein the second mount comprises a rigid material, wherein the first mount and the second mount are located along the longitudinal axis of the substrate, wherein each of the first mount and the second mount comprises a top surface, wherein the top surface comprises an indicator; a first optical marker and a second optical marker, the first optical marker configured to be attached to the top surface of the first mount, and the second optical marker configured to be attached to the top surface of the second mount, wherein the indicator provides visual guidance to a user for attaching the first mount and the second mount in a correct orientation, wherein at least one or more of the first optical marker and the second optical marker are configured to be identified by one or more optical detectors for tracking motion of the subject during the medical imaging scan.

In certain embodiments, an isolated movement of the first marker does not cause the second marker to substantially move. In certain embodiments, the first optical marker and the second optical marker are positioned such that at least one of the first optical marker and the second optical marker are viewable by one or more detectors. In certain embodiments, the central portion of the substrate comprises a narrower width than the terminal end of the substrate. In certain embodiments, one or more areas of the central portion of the substrate are removable.

In some embodiments, an optical marker apparatus for tracking motion of a subject during a medical imaging scan comprises: a substrate configured to be attached on a nose bridge of the subject of the medical imaging scan, the substrate comprising a flexible material configured to be shaped to substantially cover a curvature of the nose bridge of the subject to at least partially contact the nose bridge of the subject, the substrate comprising a longitudinal axis, wherein the substrate comprises: two terminal portion along a longitudinal axis of the substrate; and a central portion between the two terminal ends along the longitudinal axis of the substrate, wherein the central portion is configured to be more flexible than the two terminal ends; a first mount connected to a surface of the substrate, wherein the first mount is located at one of the two terminal ends of the substrate, wherein the first mount comprises a rigid material; a second mount connected to the surface of the substrate, wherein the second mount is located at the central portion of the substrate, wherein the second mount comprises a rigid material, wherein each of the first mount and the second mount comprises a top surface; a first optical marker and a second optical marker, the first optical marker configured to be attached to the top surface of the first mount, and the second optical marker configured to be attached to the top surface of the second mount, wherein at least one or more of the first optical marker and the second optical marker are configured to be identified by one or more optical detectors for tracking motion of the subject during the medical imaging scan.

In certain embodiments, the central portion of the substrate comprises a void space. In certain embodiments, the void space is configured to be formed after the substrate is attached to the nose bridge of the subject by removing some or all of the central portion. In certain embodiments, the central portion of the substrate comprises a removable portion configured to reduce a thickness of the central portion upon removal of the removable portion. In certain embodiments, the central portion of the substrate is thinner than the two terminal portions of the substrate. In certain embodiments, an isolated movement of the first marker does not cause the second marker to substantially move.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

A better understanding of the devices and methods described herein will be appreciated upon reference to the following description in conjunction with the accompanying drawings, wherein:

FIG. 1A is an anterior perspective view of an example optical marker apparatus;

FIG. 1B is an anterior perspective view of another example optical marker apparatus;

FIG. 1C is an anterior perspective view of another example optical marker apparatus;

FIG. 2A is an anterior perspective view of an example base portion;

FIG. 2B is an anterior perspective view of another example base portion;

FIG. 2C is an anterior perspective view of another example base portion;

FIG. 2D is an anterior perspective view of another example base portion;

FIG. 3A is an anterior perspective view of an example base portion;

FIG. 3B is another anterior perspective view of the example base portion of FIG. 3A;

FIG. 3C is a side perspective view of the example base portion of FIG. 3A;

FIG. 3D is a posterior perspective view of the example base portion of FIG. 3A;

FIG. 4A is an anterior perspective view of an example substrate;

FIG. 4B is an anterior perspective view of another example substrate;

FIG. 4C is an anterior perspective view of another example substrate;

FIG. 4D is an anterior perspective view of another example substrate;

FIG. 5A is an anterior perspective view of a pair of example side mounts;

FIG. 5B is an anterior perspective view of another pair of example side mounts;

FIG. 5C is an anterior perspective view of another pair of example side mounts;

FIG. 5D is an anterior perspective view of another pair of example side mounts;

FIG. 6A is an anterior perspective view of an example central mount;

FIG. 6B is an anterior perspective view of another example central mount;

FIG. 6C is an anterior perspective view of another example central mount;

FIG. 6D is an anterior perspective view of another example central mount;

FIG. 7A is a schematic diagram of a front view of an example position of one or more optical markers of an optical marker apparatus or device relative to a patient or subject of a medical imaging scan, therapeutic procedure, and/or motion tracking therefor;

FIG. 7B is a schematic diagram of a side view of an example position of one or more optical markers of an optical marker apparatus or device relative to a patient or subject of a medical imaging scan, therapeutic procedure, and/or motion tracking therefor;

FIG. 7C is a schematic diagram of a top view of an example position of one or more optical markers of an optical marker apparatus or device relative to a patient or subject of a medical imaging scan, therapeutic procedure, and/or motion tracking therefor;

FIG. 8A is a schematic diagram of a front view of an example position of one or more optical markers of an optical marker apparatus or device relative to a medical imaging scanner, therapeutic device, and/or motion tracking system or device thereof;

FIG. 8B is a schematic diagram of a side view of an example position of one or more optical markers of an optical marker apparatus or device relative to a medical imaging scanner, therapeutic device, and/or motion tracking system or device thereof;

FIG. 9A is a schematic diagram of a top view of an embodiment of rotation of an optical marker with respect to the subject;

FIG. 9B is a schematic diagram of a front view of an embodiment of rotation of an optical marker with respect to the subject;

FIG. 10 is an anterior perspective view of an example optical marker apparatus;

FIG. 11 is an anterior perspective view of an example optical marker apparatus.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

With the use of diagnostic technologies and therapeutic technologies, it can be advantageous to track for patient movement with a high degree of accuracy. Such high accuracy tracking can improve the imaging quality obtained and produced by diagnostic equipment, such as imaging technologies. Further, the use of high accuracy patient movement tracking technology can improve the application of patient therapies, such as radiation treatment, proton treatment, and the like. By accounting for patient movement with a high degree of accuracy, therapeutic technologies can apply therapies only to the targeted tissue and avoid healthy surrounding tissue.

More specifically, there are various technologies for therapeutic radiation and other therapeutics. For example, it can be advantageous in radiation therapy, proton therapy, or other therapies to dynamically apply the radiation to a targeted area in order to account for patient movement. Patient movement can include respiration, twitches or any other voluntary or involuntary movements of the patient. By dynamically and automatically tracking patient movement, radiation therapy, proton therapy, and any other kind of therapy can be applied in a more targeted way, thereby allowing surrounding healthy tissue to be avoided and/or unharmed. The systems disclosed herein can be adapted and configured to track patient translations with accuracies of about 0.1 mm and angle accuracies of about 0.1 degrees in order to better apply radiation therapy, proton therapy, or any other therapy to the targeted tissue or area of the body.

The devices, systems, and methods disclosed herein relate to a patient motion tracking system that utilizes one or more optical marker apparatuses or devices. More specifically, in certain embodiments, an optical marker apparatus or device can comprise one or more optical markers 102 which can be utilized independently and/or in connection with one or more base portions 101. In some embodiments, the one or more optical markers 102 assist the patient motion tracking system in detecting patient motion by allowing the system to more easily, accurately, and/or quickly follow and/or track movement of the patient. For example, in certain embodiments, one or more detectors and/or cameras of a patient motion tracking system and/or medical imaging scanner can be adapted to detect the location and/or movement of one or more optical markers 102 in real-time or in near real-time. In some embodiments, a patient motion tracking system can further be adapted to utilize detected locations of the one or more optical markers 102 in order to compute and/or determine movement of a patient. More specific details on different embodiments and examples of a patient motion tracking system and/or medical imaging scanner in general can be found in United States Patent Application Publication No. US 2016/0035108, which is incorporated herein by reference in its entirety and forms part of this specification.

The foregoing methodology for tracking patient movement can be applied in the diagnostic context as well as in the therapeutic context. For example, as disclosed herein, the system can be adapted to track patient movement in order to feed such movement data to an MRI scanner such that the MRI scanner can adjust the focus and position of the scanner in order to produce a clear MRI image of the patient. Further, the system can be adapted to connect to therapeutic technologies. For example, the system can be adapted to track patient movement in order to direct a therapeutic radiation beam at a diseased tissue region while avoiding surrounding healthy tissue.

The patient movement or motion tracking system, disclosed herein, can be utilized to track periodic involuntary movement of the patient, such as breathing. By tracking the periodic patient movement with a high degree of accuracy, the system can be adapted to apply a radiation therapy, a proton therapy, or the like during strategic moments when the target tissue is in a certain position while the patient's involuntary movements continue. Additionally, the system can be adapted to track not only normal breathing movement of the patient, but also the system can be adapted to track irregular movement of the patient caused by patient activity or based on diseased tissue of the patient. For example, when a patient is running, the ribs of the patient have a larger egression that the system can track in order to continuously identify a target tissue area. In another example, the patient may be suffering from COPD or other breathing disorder or diagrammatic issues. For example, the patient could be suffering from theurofusion, which is water outside the lung that prevents the patient from breathing or a tumor is irritating a lung region thereby preventing normal breathing. The system can be adapted to track such irregular patient movements due to such conditions.

Overview of Optical Marker Apparatus

In some embodiments, an optical marker apparatus or device 100 or plurality thereof can be utilized in connection with a medical imaging scanner, therapeutic device, and/or motion tracking system thereof. For example, in certain embodiments, one or more optical marker apparatus or devices 100 can be positioned on one or more regions of a patient's body subject to a medical imaging scan, therapeutic procedure, and/or motion tracking therefor. In certain embodiments, one or more detectors and/or cameras of a medical imaging scanner, therapeutic system, and/or motion tracking system can be adapted to detect the location and/or movement of such one or more optical marker apparatus or devices 100. For example, the one or more detectors can be adapted to detect the location of the one or more optical marker apparatus or devices 100 in real-time and/or in near real-time. In some embodiments, the detected location of the one or more optical marker apparatus or devices 100 can be utilized by a medical imaging system, therapeutic system, and/or motion tracking system to determine movement of a patient by detecting where the one or more optical marker apparatus or devices 100 are located. In certain embodiments, a medical imaging scanner, therapeutic system, and/or motion tracking system can be adapted to utilize such determined patient movement in obtaining and/or normalizing one or more medical images with respect to motion of the patient. Additional detail and other embodiments and examples of such medical imaging scanners and/or motion tracking systems are included in United States Patent Application Publication No. US 2016/0035108, which is incorporated herein by reference in its entirety and forms part of this specification.

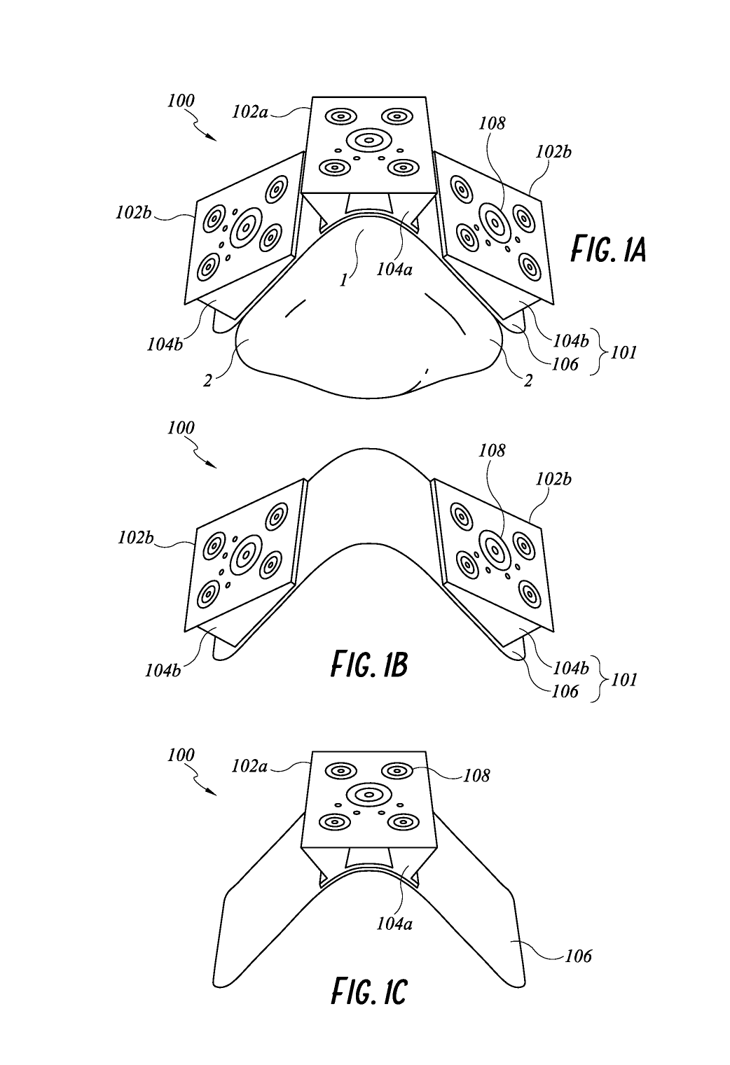

FIG. 1A is an anterior perspective view of an example optical marker apparatus. FIG. 1B is an anterior perspective view of another example optical marker apparatus. FIG. 1C is an anterior perspective view of another example optical marker apparatus. In some embodiments, an optical marker apparatus 100 can be adapted to be located and/or placed on a region of patient's body. For example, as shown in FIGS. 1A-1C, in certain embodiments, an optical marker apparatus 100 can be adapted to be placed over a nose bridge 1 of a patient. In some embodiments, an optical marker apparatus 100 can be adapted to be placed over a nose bridge 1 and/or other region of a patient's body in a generally latitudinal or generally horizontal direction with respect to a patient's face and/or other region of a patient's body. For example, in some embodiments, an optical marker apparatus 100 can be adapted to be placed along an axis substantially or nearly parallel with respect to a horizontal axis connecting a patient's eyes and/or substantially or nearly perpendicular to a vertical axis drawn between a patient's eyes or intersecting a patient's nose 1 between the nostrils. In other embodiments, an optical marker apparatus 100 can be adapted to be placed over a nose bridge 1 or other region of a patient's body in a generally longitudinal or generally vertical direction with respect to a patient's face and/or other region of a patient's body. For example, in certain embodiments, an optical marker apparatus 100 can be adapted to be placed along an axis substantially or nearly parallel with respect to a vertical axis drawn between a patient's eyes or intersecting a patient's nose between the nostrils and/or substantially or nearly perpendicular to a horizontal axis connecting a patient's eyes.

Configuring an optical marker apparatus 100 to be placed over a nose bridge 1 of a patient may be advantageous because the general shape and bone structure of a nose bridge 1 offers sufficient stability as to allow an optical marker apparatus 100 to remain in the same location in relation to the patient's body. For example, in FIGS. 1A-1C, a middle or central portion of the optical marker apparatus 100 can be adapted to be placed over a middle or central portion of a patient's nose bridge 1, such that the longitudinal axis of the optical marker apparatus 100 is perpendicular to the longitudinal axis of the patient's nose. In other embodiments, a longitudinal axis of an optical marker apparatus 100 can be adapted to be placed parallel to a longitudinal axis of a patient's nose 1.

In other embodiments, an optical marker apparatus 100 can be adapted to be placed on, over, and/or near any other portion of a patient's body. In order to provide sufficient stability as to maintain an optical marker apparatus 100 sufficiently in place for one or detectors to detect its location, it may be advantageous to configure an optical marker apparatus 100 to be placed over or near bone structure that can provide sufficient stability. For example, in certain embodiments, an optical marker apparatus 100 can be adapted to be placed over and/or near a patient's nose, chin, cheekbones, eyes, mouth, and/or forehead. Moreover, in some embodiments, an optical marker apparatus 100 can be adapted to be placed parallel to a generally longitudinal axis, parallel to a generally latitudinal axis, or along an axis that is non-parallel to either a generally longitudinal or generally latitudinal axis of a patient's face.

In some embodiments, an optical marker apparatus 100 can comprise one or more optical markers 102. In certain embodiments, an optical marker apparatus 100 can comprise a base portion 101. In some embodiments, an optical marker apparatus 100 can comprise one or more optical markers 102 attached to a base portion 101. In certain embodiments, the one or more optical markers 102 can be permanently, semi-permanently, and/or temporarily attached to a base portion 101. In some embodiments, one or more optical markers 102 can be attached to a base portion 101 via a chemical adhesive, locking or other mechanical structure, or a combination thereof. In other embodiments, the one or more optical markers 102 can be simply placed on top of a base portion 101.

The base portion 101 of an optical marker apparatus 101 can further comprise a substrate 106 and one or more mounts 104 (e.g., marker support structures, marker positioning structures, marker alignment structures, marker mounts, and/or the like). In some embodiments, markers can be integrated into the mounts 104, instead of being a separate structure attached to the mounts 104. One or more mounts 104 can be attached to the substrate 106. In certain embodiments, one or more optical markers 102 and a base portion 101 can be constructed as a single piece. In other embodiments, the one or more optical markers 102 base portion 101 can be constructed separately as two or more pieces, which can further be adapted to be attached to one another via a chemical and/or mechanical mechanism. For example, an adhesive can be used in certain embodiments. Similarly, in some embodiments, the base portion 101 can be constructed as a single piece. In other embodiments, the one or more mounts 104 and band 106 of the base portion 101 can be constructed separately as two or more pieces and can further be adapted to be attached by a chemical and/or mechanical mechanism.

Optical Marker(s)

In some embodiments, an optical marker apparatus 100 can comprise one or more optical markers 102. For example, as shown in the embodiment illustrated in FIG. 1A, an optical marker apparatus 100 can comprise three optical markers 102: two side optical markers 102B and one central optical marker 102A. However, in other embodiments, a single optical marker apparatus 100 can comprise zero, one, two, four, five, six, seven, eight, nine, or ten side and/or central optical markers 102. For example, in the embodiment illustrated in FIG. 1B, an optical marker apparatus 100 can comprise two side optical markers 102B only. In the embodiment illustrated in FIG. 1C, an optical marker apparatus 100 can comprise one central optical marker 102A only.

One or more central optical markers 102A can be configured to be attached to one or more central mounts 104A. One or more side optical markers 102B can be configured to be attached to one or more side mounts 104B. In certain embodiments, the optical marker apparatus 100 can comprise zero, one, two, three, four, five, six, seven, eight, nine, or ten side and/or central mounts 104.

In certain embodiments, one or more optical markers 102 of an optical marker apparatus 100 can be adapted to be placed along a straight line as depicted in FIGS. 1A-1C. For example, one or more side optical markers 102B and/or central optical markers 102A can be placed in a straight line along the longitudinal axis of a substrate 106 of the base portion 101. In other embodiments, however, one or more optical markers 102 of an optical marker apparatus 100 can be adapted to be placed in a staggered manner along the longitudinal axis of the substrate 106. Such configuration can be advantageous to prevent overlap of one or more optical markers 102 and/or to decrease the length and/or size of an optical marker apparatus 100. For example, such staggered configuration of one or more optical markers 102 can be advantageous when being utilized for patients with smaller or narrow noses, such as children.

In some embodiments, a medical imaging scanner, therapeutic system, and/or motion tracking system can be adapted to detect and/or track the location and/or movement of all of the optical markers 102 of one or more optical marker apparatus 100 that are placed on a patient subject to a medical imaging scan, therapy, and/or motion tracking. In other embodiments, a medical imaging scanner, therapeutic system, and/or motion tracking system can be adapted to detect the location and/or movement of only a subset of a plurality of optical markers 102 of one or more optical marker apparatus or device 100 that are placed on a patient subject to a medical imaging scan, therapeutic procedure, and/or motion tracking. For example, in some embodiments, a medical imaging scanner, therapeutic system, and/or motion tracking system can be adapted to detect the location and/or movement of one or two out of a total of three optical markers 102 of an optical marker apparatus 100. For example, in certain embodiments, a medical imaging scanner, therapeutic system, and/or motion tracking system can be adapted to detect the location and/or movement of a left and right optical marker 102B and not that of a centrally located optical marker 102A between the two. Similarly, in some embodiments, a medical imaging scanner, therapeutic system, and/or motion tracking system can be adapted to detect the location and/or movement of any subset of a central optical marker 102A and two side optical markers 102B. The visibility of one or more side markers 102B and/or central markers 102A can depend on the presence of a head coil or body coil that obstructs the view of an optical detector.

An advantage of providing two or more optical markers 102 can be to allow a motion tracking system to differentiate between rigid movement and skin movement. For example, if two or more optical markers 102 are detected to be moved together by a substantially same amount, the motion tracking system can determine that the movement was a rigid skull motion. In contrast, if only one or a subset of two or more optical markers 102 move more relative to others, then the motion tracking system can determine that the movement was an isolated elastic skin movement.

In certain embodiments, the one or more optical markers 102 can comprise one or more reference point locators 108. For example, in the embodiments shown in FIG. 1, an optical marker 102 can comprise one or more elliptical and/or circular reference point locators 108 on the surface of an optical marker 102. One or more detectors of a motion tracking system, therapeutic system, and/or medical imaging scanner can be adapted to specifically detect the one or more reference point locators 108 and/or location thereof. In other embodiments, the one or more reference point locators 108 can be rectangular, square, or of any other shape.

In some embodiments, it can be advantageous to have the one or more optical markers 102 configured such that the one or more optical markers 102 are not directly orthogonal with respect to the line of sight of one or more detectors of a medical imaging scanner, therapeutic system, and/or motion tracking system as to minimize reflection. In other embodiments, it can be advantageous to have the one or more optical markers 102 configured such that the one or more optical markers 102 are flat when viewed from one or more detectors or are directly orthogonal with respect to the line of sight of one or more detectors of a medical imaging scanner, therapeutic system, and/or motion tracking system. For example, in some embodiments, an optical marker 102 can be configured such that the angle between an orthogonal line drawn from the surface of the optical marker 102 and a line of sight from one or more detectors is about 1°, about 2°, about 3°, about 4°, about 5°, about 6°, about 7°, about 8°, about 9°, about 10°, about 11°, about 12°, about 13°, about 14°, about 15°, about 16°, about 17°, about 18°, about 19°, about 20°, about 21°, about 22°, about 23°, about 24°, about 25°, about 26°, about 27°, about 28°, about 29°, about 30°, about 35°, about 40°, about 45°, about 50°, about 55°, about 60°, about 65°, about 70°, about 75°, about 80°, about 85°, about 90°, or at an angle between any of the two angles mentioned above.

In some embodiments, the one or more mounts 104 to which the one or more optical markers 102 are attached to can be configured at particular angles to provide a desired angle of the one or more optical markers 102 relative to one or more detectors. For example, a central mount 104A can comprise two or more pillars that are shaped and angled to result in a substantially level central optical marker 102A when attached thereto. Similarly, a side mount 104B can be shaped such that a terminal end of the side mount 104B to be placed towards an end of a substrate when attached can be thicker than a central end of the side 104B to be placed near a central portion of the substrate when attached. By doing so, a desired angle of a side optical marker 102B relative to the line of sight of one or more detectors can be obtained when the side optical marker 102B is attached to such side mount 104B and when the optical marker apparatus 100 and/or substrate 106 thereof is bent or curved to be placed over the nose of a subject. More detail regarding reference point locators 108 can be found in United States Patent Application Publication No. US 2016/0035108, which is incorporated herein by reference in its entirety and forms part of this specification.

Base Portion

In certain embodiments, an optical marker apparatus 100 can comprise a base portion 101. In some embodiments, a base portion 101 of an optical marker apparatus 100 can further comprise one or more mounts 104 and a substrate 106. For example, as shown in the embodiment of FIGS. 1A-1C, one or more mounts 104 can be adapted to connect a substrate 106 of the base portion 101 with one or more optical markers 102.

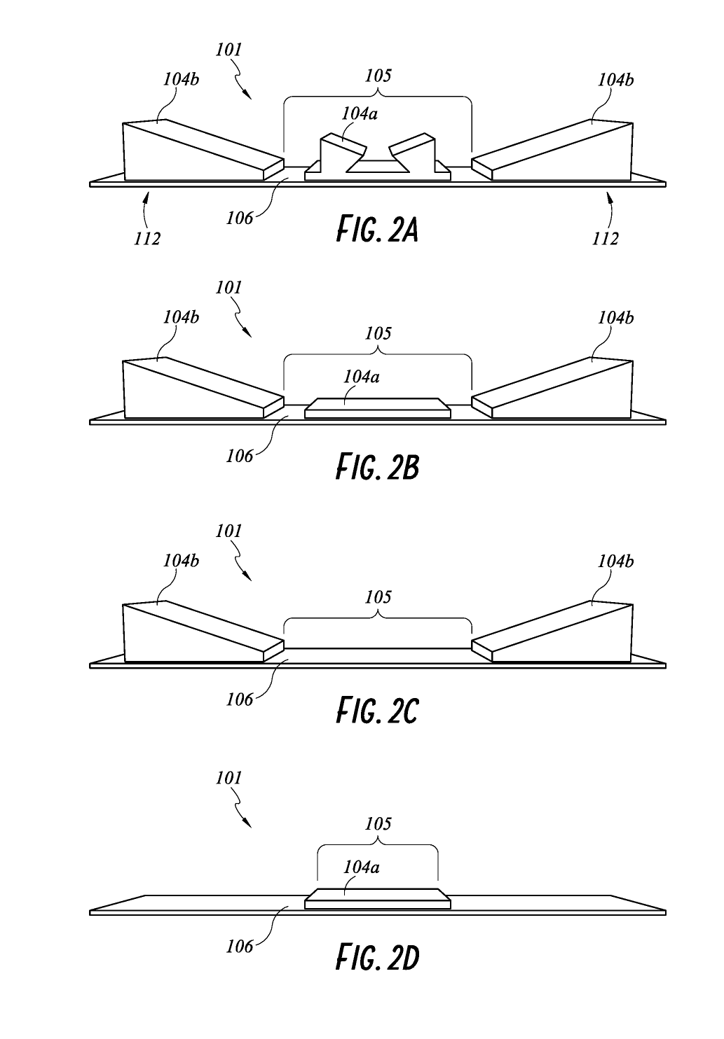

FIG. 2A is an anterior perspective view of an example base portion 101 of an optical marker apparatus 100. FIG. 2B is an anterior perspective view of another example base portion 101 of an optical marker apparatus 100. FIG. 2C is an anterior perspective view of another example base portion 101 of an optical marker apparatus 100. FIG. 2D is an anterior perspective view of another example base portion 101 of an optical marker apparatus 100.

As shown in FIGS. 2A-2D, a base portion 101 can comprise one or more mounts 104 and a substrate 106. In some embodiments, the one or more mounts 104 and one or more substrates 106 can be constructed as a single piece. In other embodiments, the one or more mounts 104 and one or more substrates 106 are constructed separately as two or more separate pieces, which can be adapted to be attached to one another via a chemical and/or mechanical mechanism. For example, in some embodiments, one or more adhesives can be used for such purpose.

In some embodiments, a base portion 101 can comprise one or more side mounts 104b that are different in configuration and/or shape from one or more central mounts 104a, as described above. In the embodiment illustrated in FIG. 2A, a base portion 101 can comprise a substrate 106 on which two side mounts 104b and one central mount 104a is attached. Each of the two side mounts 104b can be attached to the substrate 106 at or near each terminal or longitudinal end 112 of the substrate. Each of the side mounts 104b can comprise a non-uniform thickness, wherein the side mount 104b can be thicker, gradually or otherwise, towards a portion of the side mount 104b that is configured to be placed closer to a terminal end of the substrate 106. For example, as shown in FIG. 2A, in some embodiments, a top surface of a side mount 104b can be tapered or sloped such that it becomes gradually thicker towards a portion closer to a terminal end along a longitudinal axis of the substrates 106. The slope or angle of the top surface of a side mount 104b can be determined to obtain a desired angle of an optical marker 102 relative to the line of sight of one or more optical detectors when the optical marker 102 is attached to the side mount 104b. The angle or slope of a side mount 104b can be about 10°, about 20°, about 30°, about 40°, about 50°, about 60°, about 70°, about 80°, about 90°, and/or within a range defined by two of the aforementioned values. The top surface of two or more side mounts 104b can be angled such that when the substrate 106 is placed flat on a flat surface, the top surface of one side mount 104b is not parallel to the top surface of another side mount 104b. Similarly, the top surface of a side mount 104b can be angled such that it is not parallel to a side of a nose of a patient when the optical marker apparatus 100 is attached to the patient.

A portion of the base portion 101 or substrate 106 thereof between two side mounts 104b can be referred to as a central portion 105 of the base portion 101 or substrate 106 thereof. This central portion 105 can be configured to be placed over and/or in contact with a nose bridge of a subject. By placing the central portion 105 over a nose bridge, partially or wholly in contact with the nose bridge, the optical marker apparatus 101 can be held substantially in place in contact with the subject. For example, it can be advantageous to attach at least or about half of the central portion 105 of the base portion 101 or substrate 106 thereof along a longitudinal axis such that it is in contact with a rigid portion of a nose bridge of a subject.

A central mount 104a can be configured to be attached to the central portion 105. As discussed above, a central mount 104a can comprise two or more pillars or other structures that provide support for placing an optical marker 102 in a substantially flat configuration on top of the central mount 104a. For example, in the embodiment shown in FIG. 2A, a central mount 104a can comprise two pillars. When the base portion 101 and/or substrate 106 thereof is bent or otherwise deformed to match or substantially match a curvature of a subject, the central mount 104a can also be bent or otherwise deformed in a similar manner in some embodiments. The top surface of each of the two pillars can be angled or sloped downwards towards the center of the base portion 101 or substrate thereof 106. As such, when the central mount 104a is bent or deformed, the top surfaces of the two or more pillars can be leveled to provide support for attaching an optical marker 102 in a substantially flat configuration.

In other embodiments, a central mount 104a can comprise a continuously flat top surface without any pillar structures as shown in FIG. 2B. In certain such embodiments, only a central portion of a central mount 104a can be affixed to the substrate 106. Other peripheral portions of the central mount 104a, in contrast, can be simply placed above the substrate 106 without any affixation. By doing so, when the base portion 101 or substrate 106 thereof is bent or deformed for placement over a nose bridge of a subject, the peripheral portions of the central mount 104a can be separated from the substrate 106, with only a central portion of the central mount 104a being affixed to the substrate 106. This way, even when the base portion 101 or substrate 106 thereof is bent or deformed, the central mount 104a without any pillar structures can provide support for placing an optical marker 102 in a substantially flat configuration relative to the line of sight of one or more detectors.

In certain embodiments, a base portion only comprises one or more side mounts 104b and does not comprise any central mounts 104a. For example, in the embodiment shown in FIG. 2C, a base portion 101 can comprise two side mounts 104b attached to a substrate 106 without any central mounts 104a. In other embodiments, a base portion only comprises one or more central mounts 104a and does not comprise any side mounts 104b. For example, in the embodiment shown in FIG. 2D, a base portion 101 can comprise one central mount 104a attached to a substrate 106 without any side mounts 104b.

The number of mounts 104 and/or configuration thereof can be determined based on features of a medical imaging scanner, therapeutic system, and/or motion tracking system the optical marker apparatus 100 is to be used in conjunction with. For example, depending on the camera or detector positions, angles, use of a head coil, use of a body coil, and/or other features, an optical marker apparatus 100 with a base portion 101 comprising one, two, three, four, five, six, seven, eight, nine, or ten side mounts 104b and/or central mounts 104a can be utilized.

As described above, a base portion 101, substrate 106, and/or mounts 104 thereof can be bent or otherwise deformed prior to and/or during application to a subject. For example, if attaching an optical marker apparatus 100 over a nose bridge of a subject, the base portion 101, substrate 106, and/or mounts 104 of the optical marker apparatus 100 can be bent or otherwise deformed to substantially match or follow a curvature of a nose bridge or nose of the subject.

In some embodiments, the base portion 101, substrate 106, and/or mounts 104 thereof can be initially be constructed in a first configuration. For example, as shown in FIGS. 2A-2D, the substrate 106 of a base portion 101 can initially be constructed to be in a flat configuration. In certain embodiments, a substrate 106 that is initially constructed flat can later be modified as to be bent or otherwise deformed, for example to match or substantially match a curvature of a subject's skin. In some embodiments, a substrate 106 can be exposed to heat, chemicals, light, force, pressure, or any combination thereof to be molded into a non-flat shape. In certain embodiments, a substrate 106 is printed perfectly or substantially flat and is bent during a fabrication process.

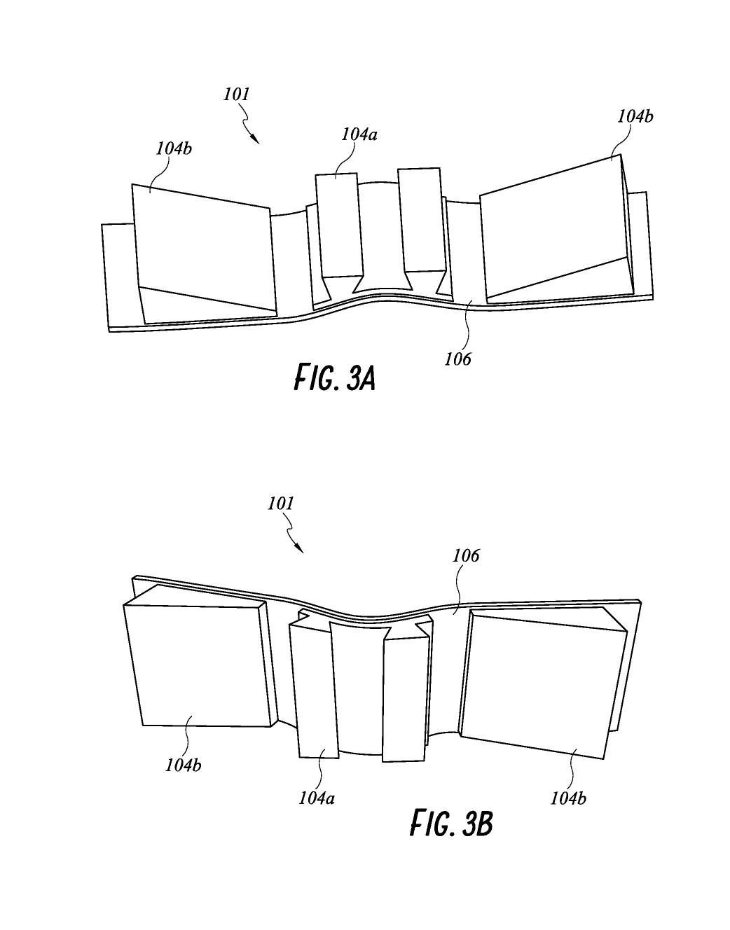

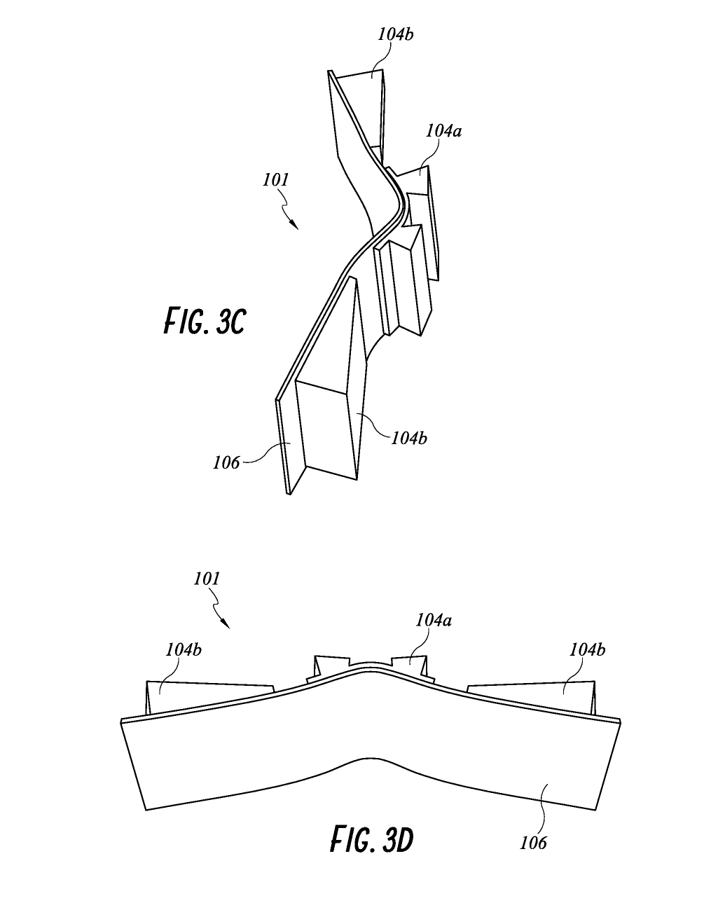

FIG. 3A illustrates an anterior perspective view of an example base portion in a deformed configuration. FIG. 3B illustrates another anterior perspective view of the example base portion of FIG. 3A. FIG. 3C illustrates a side perspective view of the example base portion of FIG. 3A. FIG. 3D is a posterior perspective view of the example base portion of FIG. 3A.

Substrate

To facilitate bending of a base portion 101, substrate 106, and/or one or more mounts 104, in some embodiments, it can be advantageous for the substrate 106, one or more mounts 104, or a portion thereof to be flexible and/or compliant. For example, in some embodiments, a flexible and/or compliant substrate 106 can be molded, bent, or otherwise modified or deformed to conform or substantially conform to the shape of the surface of a patient's skin. In some embodiments, the substrate 106 can have a malleable rigidity for flexible adjustability relative to a subject or user.

In some embodiments where an optical marker apparatus 100 is adapted to be placed over a nose bridge of a patient or subject, it can be advantageous for the base portion 101, substrate 106 and/or one or more mounts 104 to be flexible to allow the shape or configuration thereof to conform to the shape of the patient's nose bridge. This can maximize or at least increase the surface area of contact between the substrate 106 and the patient's skin as to maximize or at least increase the adhesive strength. This can also minimize the amount of slip over time and further improve the quality of one or more data gathered by a medical imaging scanner, therapeutic system, and/or motion tracking system.

In some embodiments, the substrate 106 can be adapted to dampen movement of the skin to minimize movement of one or more optical markers 102 with respect to the movement of one or more other optical markers 102 as the skin of a patient moves. As such, in some embodiments, the substrate 106, one or more mounts 104, one or more optical markers 102, and/or the optical marker apparatus 100 itself can be adapted to remain substantially in the same position on a region of a patient's body during a medical image scan, therapeutic procedure, and/or motion tracking.

In some embodiments, a substrate 106 comprises a flexible material, such as for example rubber. In certain embodiments, the structure of a substrate 106 is configured to provide flexibility. For example, in certain embodiments, the substrate 106 can be constructed with web-like material and/or fence-like material as to provide flexibility. In some embodiments, the substrate 106 can be constructed as a layer of material with a plurality of pores as to provide flexibility and/or adhesive properties when placed on a region of a patient's body. In other embodiments, the substrate 106 is constructed of a flexible material and also in a flexible structure. For example, the substrate 106 can comprise surgical tape and/or similar material.

In certain embodiments, a flexible substrate 106 can also be advantageous to allow for each of a plurality of optical markers 102 to move independently from one another. For example, if a patient squints or otherwise makes a facial expression, one or more optical markers 102 of an optical marker apparatus 100 may move. If a substrate 106 comprises non-flexible material, movement of one optical marker 102 can affect and/or cause movement of one or more other optical markers 102 of the same optical marker apparatus 100. However, if the substrate 106 is constructed of a sufficiently flexible material, movement of one optical marker 102 may not affect or at least less affect one or more other optical markers 102 that of the same optical marker apparatus 100.

As such, in some embodiments, a substrate 106 comprises certain features to reduce stress required to move one or more mounts 104 and/or optical markers 102 relative to one another. For example, in some embodiments, a plurality of optical markers 102 can be coupled to a substrate 106, directly and/or via one or more mounts 104.

In some embodiments, the width of a substrate 106 can be about 20 mm. In certain embodiments, the width of a substrate 106 can be about 10 mm, about 12 mm, about 14 mm, about 16 mm, about 18 mm, about 20 mm, about 22 mm, about 24 mm, about 26 mm, about 28 mm, about 30 mm, and/or within a range defined by two of the aforementioned values. In some embodiments, the length of a substrate 106 can be about 60 mm. In certain embodiments, the length of the substrate 106 can be about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and/or within a range defined by two of the aforementioned values. The two terminal ends of a substrate 106 can be rounded in some embodiments to provide for comfort for the subject when the optical marker apparatus 100 is attached. In other embodiments, the terminal ends of a substrate 106 can comprise sharp angles.

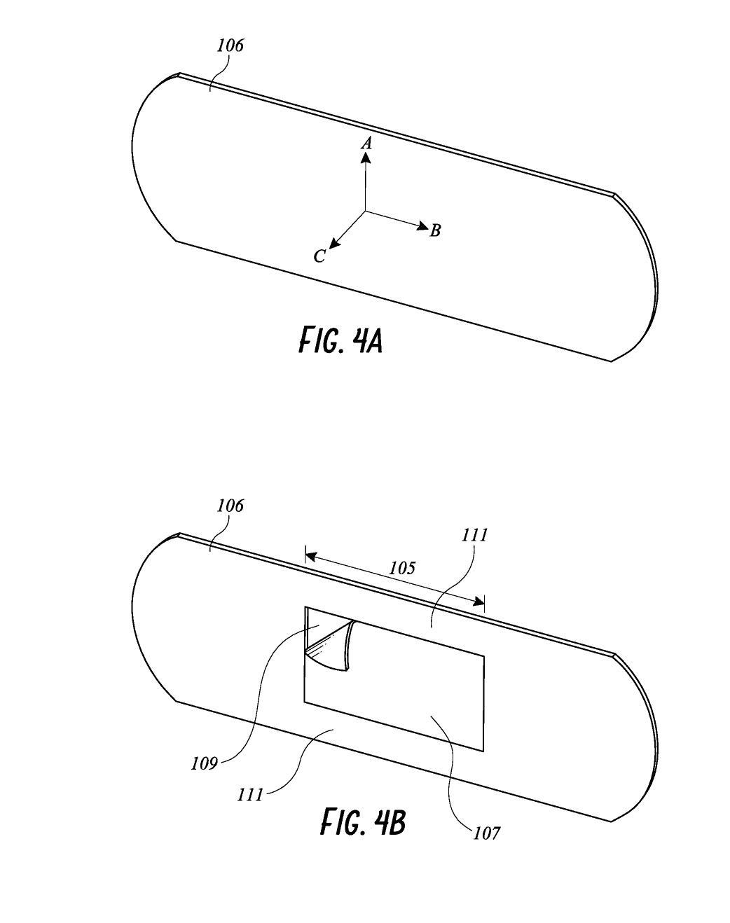

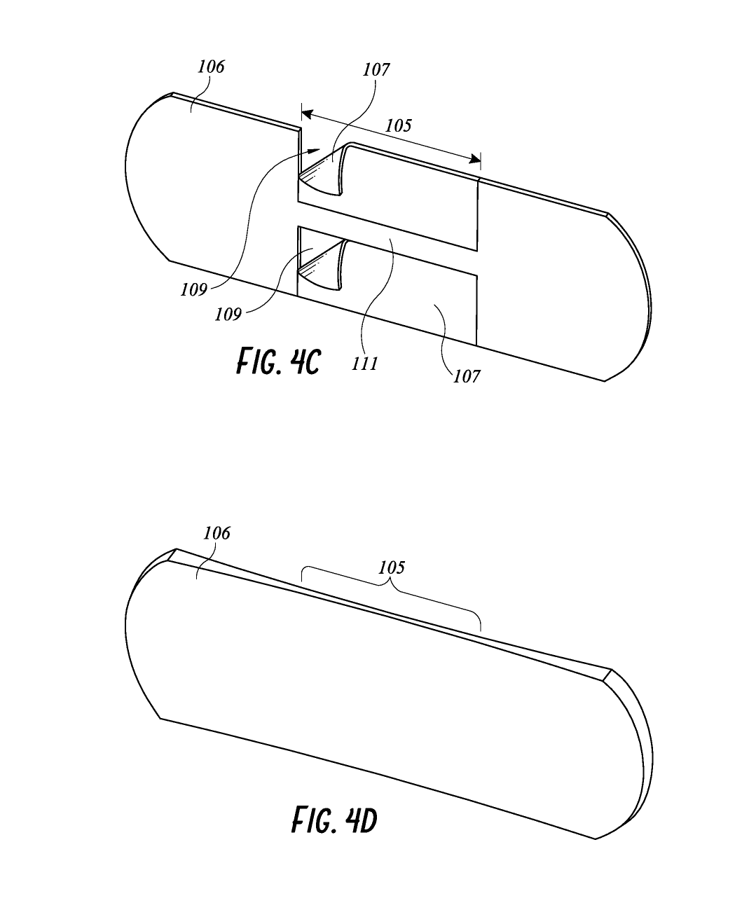

FIG. 4A illustrates an anterior perspective view of an example substrate. FIG. 4B illustrates an anterior perspective view of another example substrate. FIG. 4C is an anterior perspective view of another example substrate. FIG. 4D is an anterior perspective view of another example substrate.

In some embodiments, including the embodiment shown in FIG. 4A, a substrate 106 and/or a portion thereof, such as a central portion positioned between two or more side markers 102b can be adapted to be stretchable, bendable, compressible, and/or the like, to enable two or more side markers 102b to move at least partially independently of one another. For example, a central portion 105 as illustrated in FIGS. 4A-4B may be adapted to be stretchable, bendable, compressible, and/or the like, to enable relative motion of a plurality of side markers 102b and/or central markers 102a with respect to one another.

In some embodiments, as shown in FIG. 4A, the entire substrate 106 can comprise a substantially similar thickness, cross-sectional shape, material composition, flexibility, and/or rigidity. In other embodiments, the substrate 106 may vary in thickness, shape, material composition, flexibility, and/or rigidity along its length. For example, in some embodiments, it may be desirable to have a portion of the substrate 106 that is positioned between two or more side markers, such as side markers 102B shown in FIG. 1B, be more flexible, stretchable, and/or the like than another portion of the substrate 106, such as to enable the side markers to more easily move relative to one another after being attached to the patient. For example, in some embodiments as shown in FIG. 4D, a central portion 105 of a substrate 106 can be thinner than other areas of the substrate 106. The variance in thickness can depend on a desired amount of resistance to relative movement between the markers 102, such as two side markers 102b. In some embodiments, the difference in thickness between a central portion 105 and outer portions is a gradual change, such as a tapered change in thickness as shown in FIG. 4D. In other embodiments, the difference in thickness between a central portion 105 and outer portions may be more abrupt, such as a stepped change in thickness. For example, in some embodiments, the substrate 106 may comprise a multilayered composition formed by two or more layers of material being adhered together. In the central portion 105, the substrate 106 may comprise fewer layers than at the outer portions, thus leading to a center central portion 105 than the outer portions. As another example, the substrate 106 may be formed as a unitary structure, such as via injection molding or other manufacturing processes, and differences in thickness along the length of the substrate 106 may be part of that unitary structure's design.

In some embodiments, it may be desirable to have a central portion 105 of a substrate 106 that is configured to have a first type of rigidity, bendability, stretchability, stiffness, and/or the like before the substrate is applied to a patient, and a second type of rigidity, bendability, stretchability, stiffness, and/or the like after the substrate is applied to the patient. For example, with reference to FIG. 4A, which illustrates a coordinate system having three axes, A, B, and C, it may be desirable when applying the substrate 106 to the patient for the substrate 106 to be relatively bendable or compliant about axis A. This can enable the substrate 106 to be easily formed around the patient's nose. It may not be desirable, however, for the substrate 106 to be stretchable along a length of the substrate (e.g., in a direction parallel with axis B) when applying the substrate 106 to the patient's nose. Further, it may be desirable for the substrate 106 to be configured to resist rotation of the markers relative to each other about axis C when the substrate 106 is being applied to the patient's nose. In other words, it may be desirable to enable two or more markers to rotate with respect to one another about axis A when the substrate 106 is being applied to a patient's nose, and it may be desirable to limit other movements of the markers with respect to one another when the substrate 106 is being applied to the patient's nose. After the substrate 106 has been applied to the patient's nose, however, it may be desirable to allow the markers to move more freely with respect to one another. This may, for example, enable the motion tracking system to detect relative motion of the markers with respect to one another and determine that such motion is due to localized skin movement and not necessarily movement of the object of interest, such as the patient's brain.

In certain embodiments, such as shown in FIGS. 4B and 4C, changing the rigidity, flexibility, stretchability, and/or the like of the substrate 106 can be accomplished by one or more portions of the substrate 106 comprising a void space (e.g., void, space, opening, reduced cross-sectional area portion, and/or the like) configured to be opened after application of the substrate 106 to the patient. For example, in the embodiment shown in FIG. 4B, part of the central portion 105 can comprise a void space 109, resulting in the two terminal portions of the substrate 106 being connected by two longitudinally extending portions 111. The void space 109 may be created after applying the substrate 106 to the patient by removing one or more removable portions 107. The removable portion 107 may, for example, be attached to the rest of the substrate 106 at a score line, perforated region, and/or the like that enables the removable portion 107 to be easily and consistently pulled apart from the rest of the substrate 106 without damaging the rest of the substrate 106. In some embodiments, the removable portion 107 may comprise one or more pull tabs that enable a user to more easily get a hold of the removable portion 107 to remove it after application to a patient. Although this and some other embodiments disclosed herein illustrate a removable portion 107 creating a void 109 that passes completely through the substrate 106, some embodiments may comprise a removable portion 107 that may be configured to remove only a portion of the material of the substrate 106 at that area, and thus not create a void 109 that passes completely through the thickness of the substrate 106. For example, the substrate 106 may comprise a composite multilayered structure having at least one layer that is more stretchable, bendable, and/or flexible than a second layer. In such an embodiment, it may be desirable for the removable portion 107 to cause removal of the second layer, while leaving the first layer that is more stretchable, bendable, and/or flexible. In the embodiment shown in FIG. 4C, the central portion 105 can comprise two void spaces 109, created by removal of two removable portions 107, resulting in the two terminal portions of the substrate 106 being connected by a single bridge-like longitudinally extending portion 111. In some embodiments, a central portion 105 is configured to be completely removable, thus separating the substrate into two separate non-connected portions after application to the patient.

In certain embodiments, the one or more void spaces along the central portion 105 can be configured to formed after attaching the substrate 106 to a subject. In other words, initially the substrate 106 can comprise a continuous band of material to assist attachment of the substrate 106 on a subject. After attaching the substrate 106, one or more portions of the central 105 can then be removed from the remainder of the substrate 106, thereby resulting in less material along the central portion 105 to decrease the effect of movement of one terminal end or side marker 102b on another terminal end or side marker 102b. For example, in the embodiment shown in FIG. 4B, a single void space can be formed in the center of central portion 105 by removing a cut-out portion thereof. Similarly, in the embodiment shown in FIG. 4C, one or two void spaces can be formed along the top and bottom of the central portion 105 by removing one or two cut-out portions.

In some embodiments, the substrate 106 or a portion thereof may be adapted to be stiff enough that the optical marker apparatus 100 can be easily mounted to a patient as a single unit, but flexible enough to present minimal resistance to relative motion of the markers while installed on the patient. In some embodiments, the substrate 106 is constructed from a heat-sensitive material that softens and/or becomes more flexible when exposed to heat, such as body heat. This may make the unit easier to install on the patient, and allow less resistance to relative motion of the markers 102 once the substrate 106 has softened due to exposure to body heat from skin contact with the patient.

In some embodiments, a central portion 105 of the substrate 106 can be adapted to be dissolvable after application to the patient or can be adapted to be removed, cut, separated, and/or the like after application to the patient. For example, in some embodiments, the central portion 105 may comprise one or more score lines, perforated areas, and/or the like that make it relatively easy to remove or disconnect at least a portion of the central portion 105 after application to the patient. In some embodiments, a central portion 105 is adapted to be removable, to increase flexibility of the central portion 105 after application to the patient, but another portion is adapted to remain in place, so that the entire apparatus 100 can still be removed as a single unit after the medical procedure is completed. For example, an oval- or other-shaped portion may be adapted to be removable from the central portion 105.

In some embodiments, as mentioned above, it may be advantageous to construct a substrate 106 with sufficiently rigid material as to provide ease when attaching or otherwise placing the optical marker apparatus 100 on a patient. For example, if material used to construct the substrate 106 is too flexible, then it may be difficult for a healthcare provider to place an optical marker apparatus 100 on a patient. As such, there may be an optimal range of flexibility and/or rigidity of the material used to construct the substrate 106 as to provide sufficient support for easy application and also allow each of the optical markers 102 to move independently to a certain degree without substantially affecting one or more other optical markers 102.

In certain embodiments, one or more surfaces of a substrate 106 can be adapted to be placed or otherwise attached to a patient's skin. In some embodiments, the substrate 106 can be adapted to be attached to a patient's skin via one or more adhesives. Any medical adhesive can be used.

In certain embodiments, it can be advantageous for the substrate 106 to be constructed of a material and/or in a manner such that the surface area of contact between the substrate 106 and a patient's skin is maximized or is at least higher than that from using a continuously flat substrate 106. In some such embodiments where a substrate 106 comprises a web-like material and/or structure, water can be used as an adhesive. For example, in certain embodiments, when some amount of water is applied to the skin of a patient and a substrate 106 is placed such that a certain amount of water is stuck between the substrate 106 and a patient's skin, the material and/or structure of the substrate can be constructed in such a manner as to utilize the surface tension of the water can provide ample support and/or stability for a substrate 106, the one or more mounts 104, and/or one or more optical markers 102.

In certain embodiments, the substrate 106 can comprise rubber, rubber-like material, or any other flexible material. In some embodiments, the substrate 106 is made of a thin layer of rubber or other flexible material. In certain embodiments, the thickness of the substrate 106 can be about 0.01 mm, about 0.02 mm, about 0.03 mm, about 0.04 mm, about 0.05 mm, about 0.06 mm, about 0.07 mm, about 0.08 mm, about 0.09 mm, about 0.10 mm, about 0.20 mm, about 0.30 mm, about 0.40 mm, about 0.50 mm, about 0.60 mm, about 0.70 mm, about 0.80 mm, about 0.90 mm, about 1 mm, about 2 mm, about 3 mm, about 4 mm, about 5 mm, about 6 mm, about 7 mm, about 8 mm, about 9 mm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, or between any two numbers mentioned above.

In certain embodiments, the substrate 106 is sufficiently thick as to provide ample support for one or more mounts 104 and/or one or more optical markers 102. For example, in some embodiments, the substrate 106 is sufficiently thick as to allow the one or more mounts 104 and/or one or more optical markers 102 as to remain substantially in the same position with respect to a patient's skin despite movement of a patient. In other words, in certain embodiments, the one or more optical markers 102 and/or mounts 104 can be affixed or attached to a substrate 106 in a manner such that the substrate 106 moves naturally according to movement of the patient skin.

Mounts

In some embodiments, an optical marker apparatus 100 comprises one or more mounts 104. In certain embodiments, one or more mounts 104 can be adapted to connect and/or attach a substrate 106 with one or more optical markers 102. In certain embodiments, a substrate 106, one or more mounts 104, one or more optical markers 102, and/or any combination thereof can be constructed of a single piece of material. In other embodiments, a substrate 106, one or more mounts 104, one or more optical markers 102, and/or any combination thereof can be constructed separately and adapted to be attached and/or otherwise connected afterwards via a chemical and/or mechanical mechanism, such as for example using an adhesive.

In some embodiments, one or more optical markers 102 can be adapted to be placed and/or otherwise attached onto a top surface of one or more mounts 104. In certain embodiments, one or more optical markers 102 can be adapted to be attached to a surface of one or more mounts 104 via a chemical and/or mechanical mechanism, such as via an adhesive.

In some embodiments, one or more mounts 104 can be constructed of a rigid material (and/or, in some embodiments, material that is more rigid than the substrate 106). For example, in certain embodiments, the one or more mounts 104 can be constructed of polylactic acid plastic, ABS, nylon, any type of thermoplastic, foam, or any other material that is relatively rigid to provide sufficient support to one or more attached optical markers 102. In some embodiments, it may be advantageous to use a type of thermoplastic with a sufficiently low melting point to construct a mount 104.

In certain embodiments, an angle of a top surface of one or more mounts 104 can determine the angle at which one or more optical markers 102 are placed. For example, one or more side mounts 104b can comprise a top surface that is slanted or angled. In certain embodiments, one or more optical markers 102 can be attached or otherwise placed onto such surfaces; as such, the angle of the top surface of a mount 104 can determine an angle of the optical marker 102.

The shape of a mount 104 and/or angle of any surface of a mount 104 can be of any shape and/or angle as to obtain a desired angle of an optical marker 102. In some embodiments, the angle of one or more optical markers 102 can be determined by the angle and/or shape of a mount 104 and/or the angle and/or shape of a substrate 106. It may be advantageous in certain embodiments to coordinate the particular shapes and/or angles of a substrate 106 and one or more mounts 104 as to obtain a desirable angle of one or more optical markers 102.

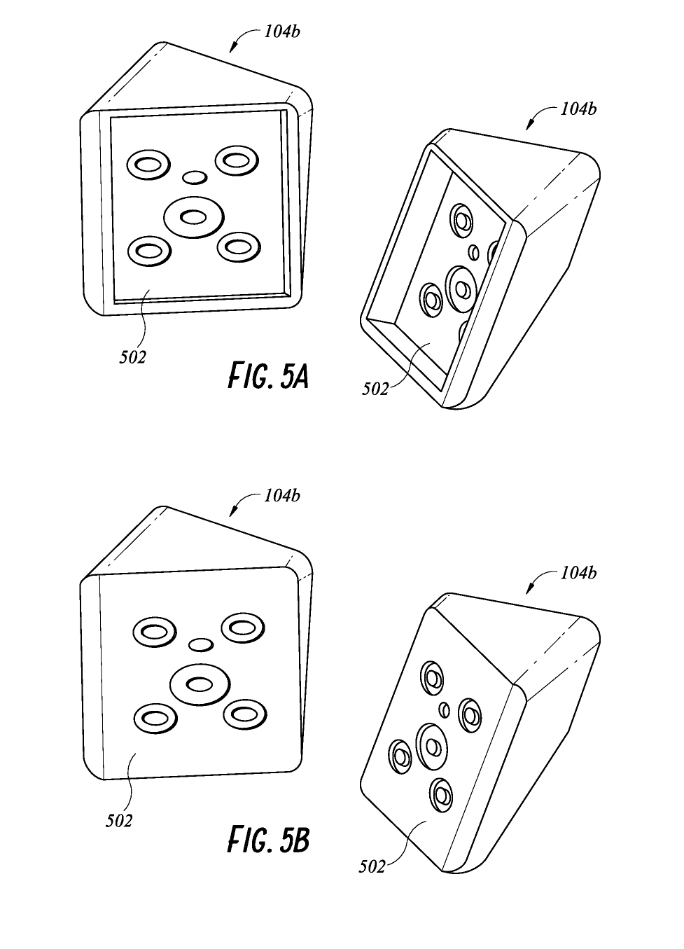

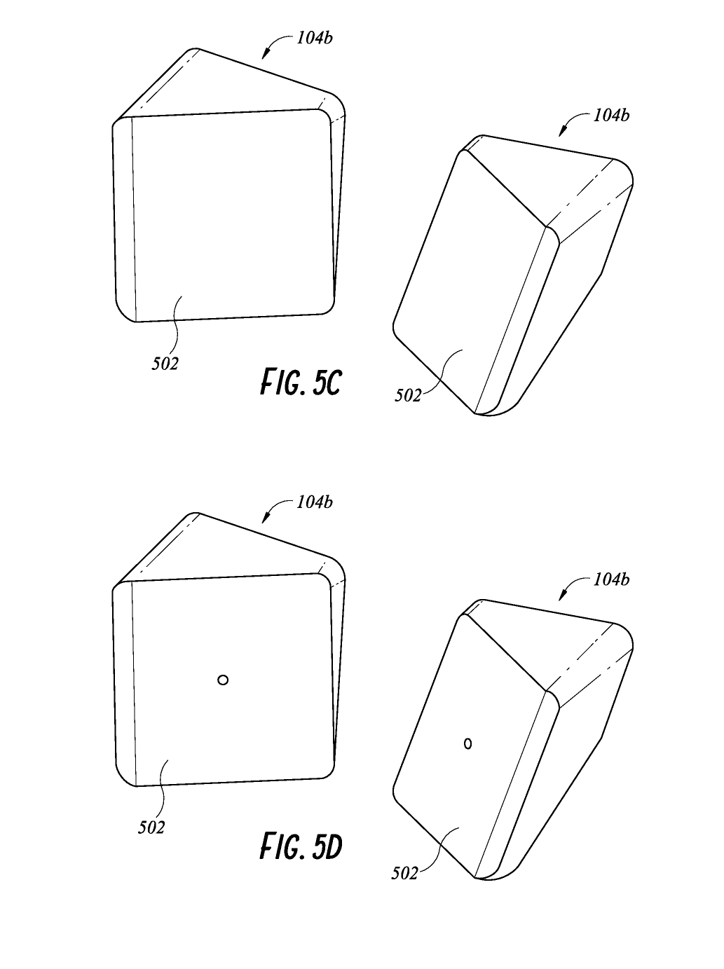

FIG. 5A illustrates an anterior perspective view of a pair of example side mounts. FIG. 5B illustrates an anterior perspective view of another pair of example side mounts. FIG. 5C illustrates an anterior perspective view of another pair of example side mounts. FIG. 5D illustrates an anterior perspective view of another pair of example side mounts.

In some embodiments, a side mount 104b can comprise a recessed top surface 502. For example, as shown in FIG. 5A, a top surface of a side mount 104b can be recessed such that an optical marker 102 can be inserted into the top surface 502. A recessed area of a top surface can be about 15 mm across. In other embodiments, a width and/or length of a recessed area of a top surface can be about 10 mm, about 11 mm, about 12 mm, about 13 mm, about 14 mm, about 15 mm, about 16 mm, about 17 mm, about 18 mm, about 19 mm, about 20 mm, and/or within a range defined by two of the aforementioned values.

The top surface 502 can comprise one or more elliptical shapes or other pattern that substantially matches one or more elliptical shapes or other pattern of an optical marker 102 to be attached thereto. This can guide a user to attach the optical marker 102 in a particular orientation and/or direction. For example, an arrow or other indicia can be provided on the top surface 502. The one or more patterns or other indications can provide a visual guide for a user to attach an optical marker 102 onto a mount according to the correct top and bottom orientation for example. The one or more elliptical shapes or pattern can be etched on the top surface 502 in some embodiments, for example by a laser printer onto a mount made of plastic.

In other embodiments, the top surface 502 can be substantially flat instead of being recessed as illustrated in FIG. 5B. A flat top surface 502 can similarly comprise one or more elliptical shapes, arrow, or other patterns or indicia to guide a user to attach an optical marker in a particular orientation or direction. Because the top surface 502 is flat in some embodiments, an optical marker 102 can be glued on or otherwise attached instead of being placed in a recessed area. This can be advantageous from a manufacturing perspective because it can be difficult to manufacture a recessed area of a top surface 502 that substantially matches the shape or contours of an optical marker.

In certain embodiments, the top surface 502 may not comprise any patterns or other indications as shown in FIG. 5C. In some embodiments, the top surface 502 can comprise one or more holes or recessed portions as shown in FIG. 5D for facilitating attachment on an optical marker. For example, an optical marker 102 can comprise one or more protruding portions extending from a back or bottom surface of the optical marker 102. Such one or more protrusions can be configured to be inserted into the one or more holes or recessed portions of the top surface 502 to attach an optical marker 102 to a mount 104.

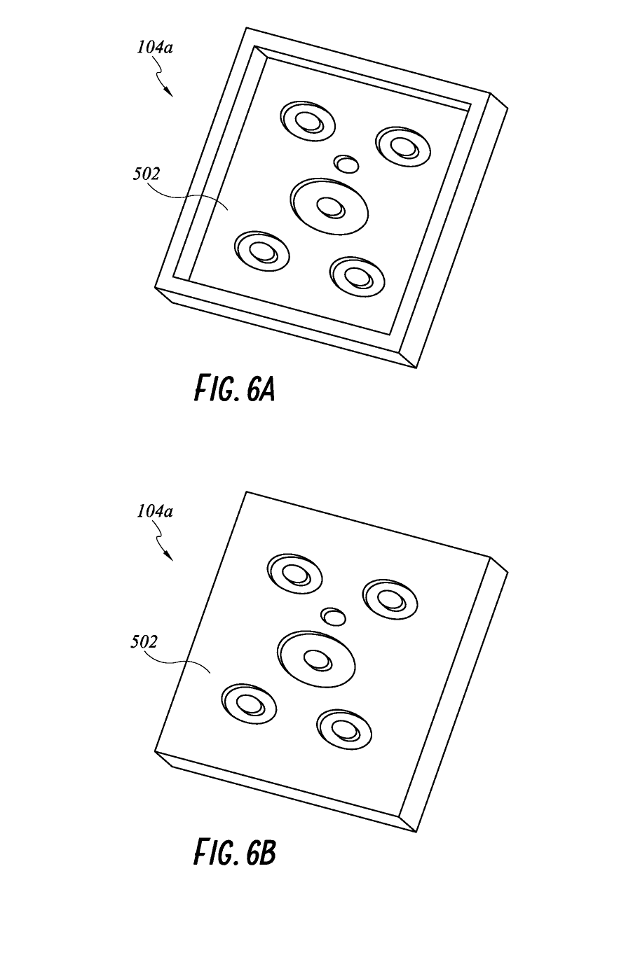

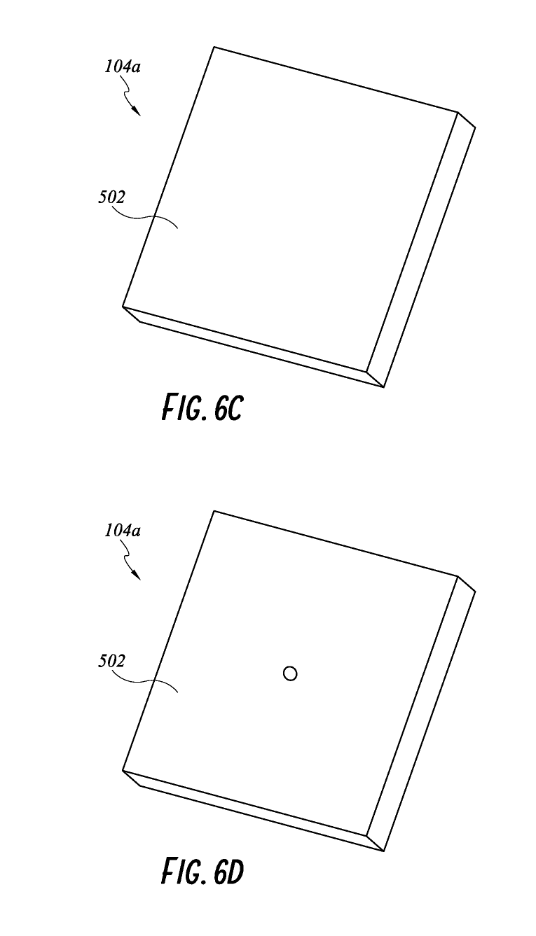

FIG. 6A illustrates an anterior perspective view of an example central mount. FIG. 6B is an anterior perspective view of another example central mount. FIG. 6C is an anterior perspective view of another example central mount. FIG. 6D is an anterior perspective view of another example central mount.

In some embodiments, similar to the side mounts 104b, the top surface 502 a central mount 104a can also be recessed. The top surface 502 of a central mount 104a can also comprise one or more patterns or indications, such as one or more elliptical patterns as shown in FIG. 6A. Moreover, in certain embodiments, similar to the side mounts 104b, the top surface 502 of a central mount 104a can also be substantially flat but still comprise one or more patterns or indications as shown in FIG. 6B. Further, in certain embodiments, similar to the side mounts 104b, the top surface 502 of a central mount 104a can be substantially flat without any patterns as shown in FIG. 6C. In addition, in some embodiments, similar to the side mounts 104b, the top surface 502 of a central mount 104a can also comprise one or more holes or recessed areas to which one or more protrusions of an optical marker 102 can be inserted into.

Optical Marker Apparatus of Different Sizes

In some embodiments, it can be advantageous to provide optical marker apparatus 100 that are adjustable and/or of different sizes. For example, optical markers apparatus or devices 100 of different sizes can be advantageous to accommodate for children and/or adults with noses and/or different facial features of different sizes where an optical marker apparatus 100 is to be attached. In some embodiments, one or more optical markers 102, one or more mounts 104, and/or one or more substrates 106 can be adapted to be of a different size for such purpose. For example, some embodiments of the optical marker apparatus 100 can comprise a longer substrate 106 than others.

In some embodiments, an optical marker system can comprise optical marker apparatus or devices 100 of just two sizes: a large and a small. It can be advantageous to provide two sizes to accommodate for children or others with smaller noses or other facial features to which an optical marker apparatus 100 is to be attached. For example, optical marker apparatus or devices 100 or substrates 106 thereof of different sizes can accommodate subject's with steeper noses or shallower noses with a lower ridge. At the same time, however, the imaging process may be overly complicated if optical marker apparatus 100 of too many different sizes are offered. In certain embodiments, a system can comprise optical marker apparatus 100 of three sizes: a large, medium, and small.

In some embodiments, an optical marker apparatus 100 can be adjustable as to accommodate for imaging subjects with noses or other facial features of different sizes to which an optical marker apparatus 100 is to be attached. For example, in certain embodiments, a substrate 106 can be sufficiently flexible as to be molded according to the curvature of the skin of a patient. In some embodiments, one or more mounts 104 can also be sufficiently flexible to be molded as to accommodate for different angles resulting from the curvature of a patient's skin. In yet other embodiments, optical markers 102 of different sizes and/or shapes can be utilized to accommodate differences in the patient. Any or all of the adjustable features above or a subset thereof can also be combined as well to provide an optical marker apparatus 100 that is adjustable to the particular nature of a patient.

In certain embodiments, a substrate 106, one or more mounts 104, one or more optical markers 102, any subset thereof, or all of the above can be adjusted according to the particulars of a patient in order to obtain an acceptable angle of one or more optical markers 102. For example, in some embodiments, it can be advantageous to have the one or more optical markers 102 configured such that the one or more optical markers 102 are not directly orthogonally placed with respect to the line of sight of one or more detectors of a medical imaging scanner, therapeutic device, and/or motion tracking system as to minimize reflection. For example, in some embodiments, an optical marker 102 can be configured such that the angle between an orthogonal line drawn from the surface of the optical marker 102 and a line of sight from one or more detectors is about 1°, about 2°, about 3°, about 4°, about 5°, about 6°, about 7°, about 8°, about 9°, about 10°, about 11°, about 12°, about 13°, about 14°, about 15°, about 16°, about 17°, about 18°, about 19°, about 20°, about 21°, about 22°, about 23°, about 24°, about 25°, about 26°, about 27°, about 28°, about 29°, about 30°, about 35°, about 40°, about 45°, about 50°, about 55°, about 60°, about 65°, about 70°, about 75°, about 80°, about 85°, about 90°, or at an angle between any of the two angles mentioned above.

Optimal Marker Position

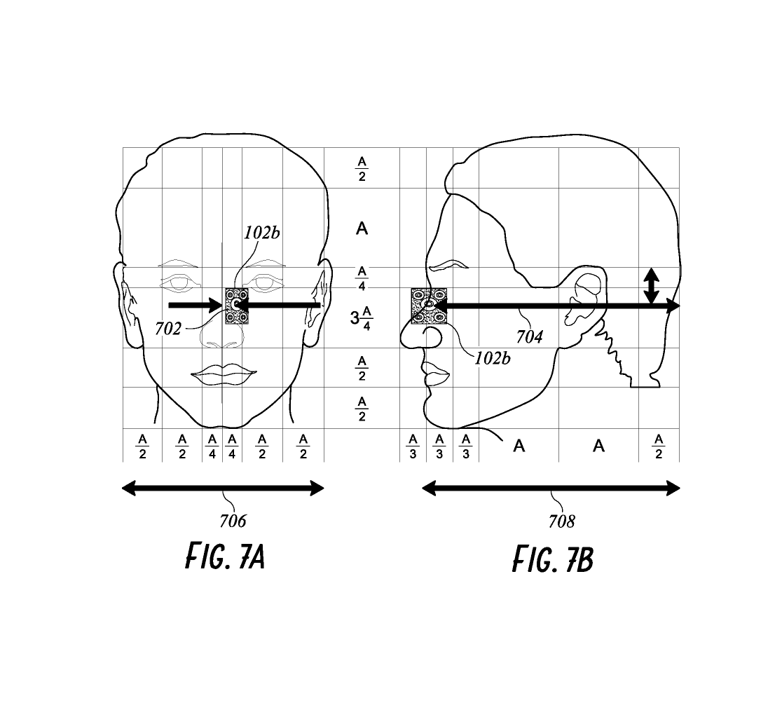

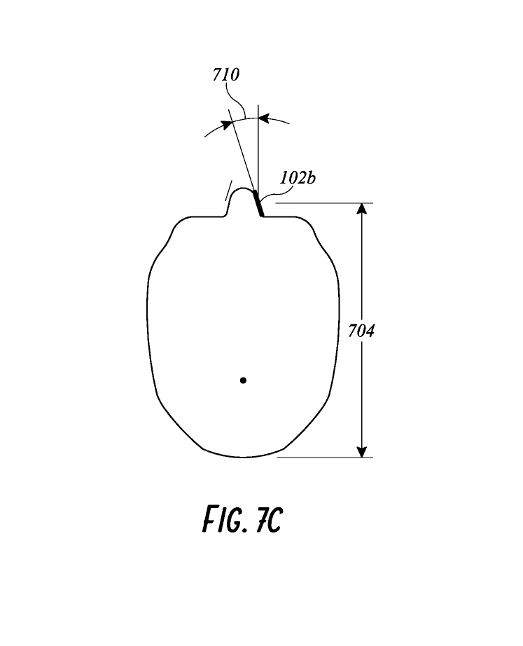

As discussed herein, the position of one or more optical markers 102 can be crucial in obtaining quality tracking data by detecting and/or tracking the one or more optical markers 102. FIG. 7A illustrates a schematic diagram of a front view of an example position of one or more optical markers of an optical marker apparatus or device relative to a patient or subject of a medical imaging scan, therapeutic procedure, and/or motion tracking therefor. FIG. 7B illustrates a schematic diagram of a side view of an example position of one or more optical markers of an optical marker apparatus or device relative to a patient or subject of a medical imaging scan, therapeutic procedure, and/or motion tracking therefor. FIG. 7C illustrates a schematic diagram of a top view of an example position of one or more optical markers of an optical marker apparatus or device relative to a patient or subject of a medical imaging scan, therapeutic procedure, and/or motion tracking therefor.

As illustrated in FIG. 7A, a cranial base width 706 of a subject can be generally about 142 mm. For some subjects, the cranial base width 706 can be as wide as about 150 mm and can be as narrow as about 136 mm. Further, as illustrated, in some embodiments, one or more side optical markers 102b can be configured to be placed on the side of the subject's nose 2. More specifically, by placing a side optical marker 102b on the side of a subject's nose, a horizontal distance 702 along the cranial width between the center of the subject's nose and the center of the side optical marker 102b can be specified to pinpoint the position of the side optical marker 102b. This horizontal distance 702 along the cranial width can be about 11 mm in some embodiments. In certain embodiments, this horizontal distance 702 along the cranial width can be about 5 mm, about 6 mm, about 7 mm, about 8 mm, about 9 mm, about 10 mm, about 11 mm, about 12 mm, about 13 mm, about 14 mm, about 15 mm, and/or within a range defined by two of the aforementioned values.

As illustrated in FIG. 7B, a cranial length 708 of a subject can be generally about 191 mm. For some subjects, the cranial length 708 can be as wide as about 200 mm and can be as narrow as about 184 mm. In some embodiments, by placing an optical side marker 102b on the side of a subject's nose 2, a horizontal distance 704 along the cranial length between the back of the head or back end of the cranial length and the center of the side optical marker 102b can be specified to pinpoint the position of the side optical marker 102b. This horizontal distance 704 along the cranial length can be about 188 mm in some embodiments. In certain embodiments, this horizontal distance 704 along the cranial length can be about 170 mm, about 175 mm, about 180 mm, about 185 mm, about 190 mm, about 195 mm, about 200 mm, and/or within a range defined by two of the aforementioned values.

As illustrated in FIG. 7C, placement of a side optical marker 102b on the side of a subject's nose 2 can result in an angle 710 between the surface of the marker 110 and a vertical line when viewed from the top of the subject's head. This angle 710 can be further used to pinpoint the configuration or position of a side optical marker 102b. The angle 710 between a line extended along the surface of the side optical marker 102b and a vertical line when viewed from the top of the subject's head as illustrated in FIG. 7C can be about 17°. In certain embodiments, this angle 710 can be about 10°, about 11°, about 12°, about 13°, about 14°, about 15°, about 16°, about 17°, about 18°, about 19°, about 20°, about 21°, about 22°, about 23°, about 24°, about 25°, about 26°, about 27°, about 28°, about 29°, about 30°, about 35°, about 40°, about 45°, about 50°, about 55°, about 60°, about 65°, about 70°, about 75°, about 80°, about 85°, about 90°, and/or within a range defined by two of the aforementioned values. This angle 710 can depend on the shape or configuration of a subject's nose.

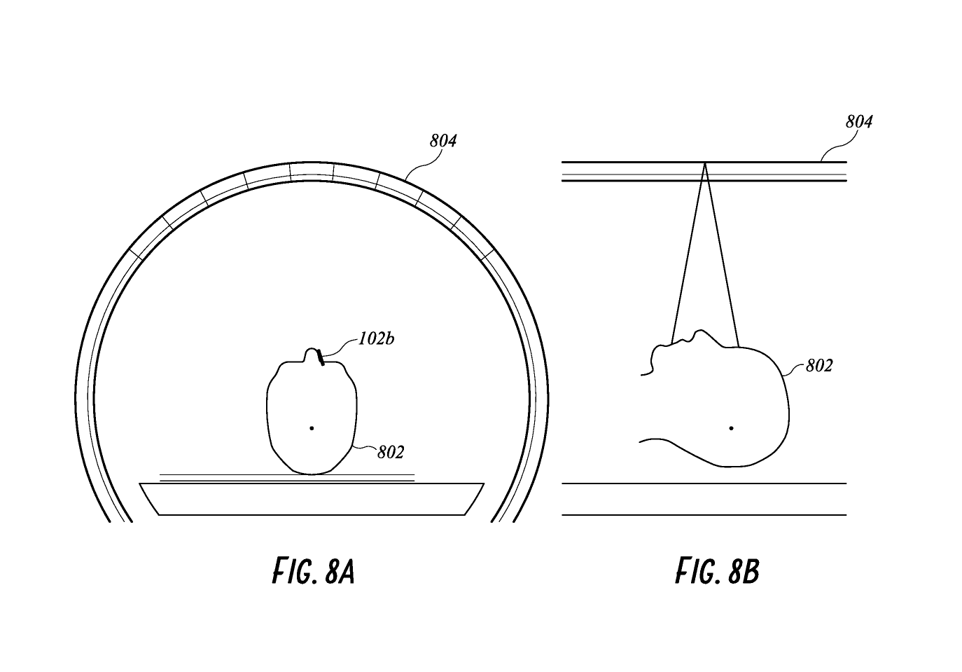

Also, optimal positioning of one or more optical markers 102 can depend on the type of medical imaging scanner, therapeutic device, and/or motion tracking system being utilized and/or the specific location and/or angle of one or more detectors or cameras thereof as depicted in FIGS. 8A-8B. FIG. 8A illustrates a schematic diagram of a front view of an example position of one or more optical markers of an optical marker apparatus or device relative to a medical imaging scanner, therapeutic device, and/or motion tracking system or device thereof. FIG. 8B illustrates a schematic diagram of a side view of an example position of one or more optical markers of an optical marker apparatus or device relative to a medical imaging scanner, therapeutic device, and/or motion tracking system or device thereof.

As shown in FIGS. 8A and 8B, the motion of a subject 802 undergoing a medical imaging scan and/or therapeutic device can be obtained by utilizing an optical marker apparatus. For example, one or more side optical markers 104b can be attached to a subject 802 of a medical imaging scan or therapeutic procedure using one or more optical marker apparatus of devices 100. The one or more side optical markers 104b and/or central markers 104a can be detected and tracked using one or more optical detectors and/or cameras of a medical imaging scanner or therapeutic system 804.

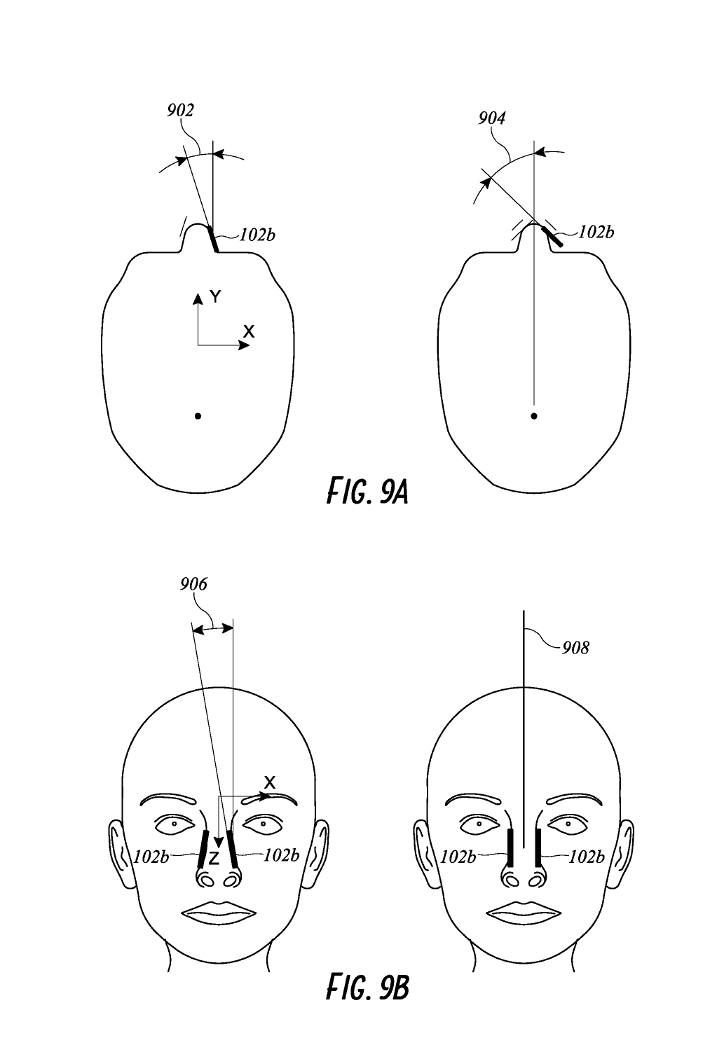

Moreover, optimal positioning of one or more optical markers 102 can also depend on a rotational characteristic of one or more optical markers 102. FIG. 9A illustrates a schematic diagram of a top view of an embodiment of rotation of an optical marker with respect to the subject. FIG. 9B illustrates a schematic diagram of a front view of an embodiment of rotation of an optical marker with respect to the subject.

As illustrated in FIGS. 9A and 9B, a side optical marker 102b can be angled in line with the physiological angle of the subject's nose. More specifically, an angle 902 can exist between a vertical line drawn along the y axis, as illustrated in FIG. 9A, and a line extending along the side optical marker 102b. This angle 902 can be about 17° for example. In such embodiments, a camera or detector entrance angle viewing the subject from the top of the subject's head as illustrated in FIG. 9A may range between about +3° to about +59°.

In certain embodiments, each of the angle 902 and a camera or detector entrance angle viewing the subject from the top of the subject's head as illustrated in FIG. 9A can be about 0°, about 5°, about 10°, about 15°, about 20°, about 25°, about 30°, about 35°, about 40°, about 45°, about 50°, about 55°, about 60°, about 65°, about 70°, about 75°, about 80°, about 85°, about 90°, and/or within a range defined by two of the aforementioned values.

In other embodiments, an optical marker apparatus or device 100 can alter or manipulate the angle 904 between the vertical line along the y axis and a line extending from the surface of the side optical marker 102b, as illustrated in FIG. 9A. For example, the angle 904 may be about 45° in some embodiments. Due to such configuration, the camera or detector entrance angle viewing the subject from the top of the subject's head as illustrated in FIG. 9A may range between −25° to +31° in some embodiments.

In certain embodiments, each of the angle 904 and the camera or detector entrance angle viewing the subject from the top of the subject's head as illustrated in FIG. 9A can be about −90°, about −85°, about −80°, about −75°, about −70°, about −65°, about −60°, about −55°, about −50°, about −45°, about −40°, about −35°, about −30°, about −25°, about −20°, about −15°, about −10°, about −5°, about 0°, about 5°, about 10°, about 15°, about 20°, about 25°, about 30°, about 35°, about 40°, about 45°, about 50°, about 55°, about 60°, about 65°, about 70°, about 75°, about 80°, about 85°, about 90° and/or within a range defined by two of the aforementioned values.

Similarly, in certain embodiments, an angle 906 may be exist between an extended line along a side optical marker 102b and a vertical line along the z axis when viewed from a front view as illustrated in FIG. 9B. For example, the angle 906 may be about 10°. In such embodiments, a camera or detector entrance angle viewing the subject from above the subject's face as illustrated in FIG. 9B may be about 10°.

In certain embodiments, each of the angle 906 and camera or detector entrance angle viewing the subject from above the subject's face as illustrated in FIG. 9B can be about 0°, about 5°, about 10°, about 15°, about 20°, about 25°, about 30°, about 35°, about 40°, about 45°, about 50°, about 55°, about 60°, about 65°, about 70°, about 75°, about 80°, about 85°, about 90°, and/or within a range defined by two of the aforementioned values.

In other embodiments, an optical marker apparatus or device 100 can alter or manipulate the angle 908 between an extended line along a side optical marker 102b and a vertical line along the z axis when viewed from a front view as illustrated in FIG. 9B. For example, the angle 908 may be about 0° in some embodiments. Due to such configuration, the camera or detector entrance angle viewing the subject from above the subject's face as illustrated in FIG. 9B may be about 0° in some embodiments.

In certain embodiments, each of the angle 908 and the camera or detector entrance angle viewing the subject from the top of the subject's head as illustrated in FIG. 9B can be about −90°, about −85°, about −80°, about −75°, about −70°, about −65°, about −60°, about −55°, about −50°, about −45°, about −40°, about −35°, about −30°, about −25°, about −20°, about −15°, about −10°, about −5°, about 0°, about 5°, about 10°, about 15°, about 20°, about 25°, about 30°, about 35°, about 40°, about 45°, about 50°, about 55°, about 60°, about 65°, about 70°, about 75°, about 80°, about 85°, about 90° and/or within a range defined by two of the aforementioned values.

### OTHER EMBODIMENTS

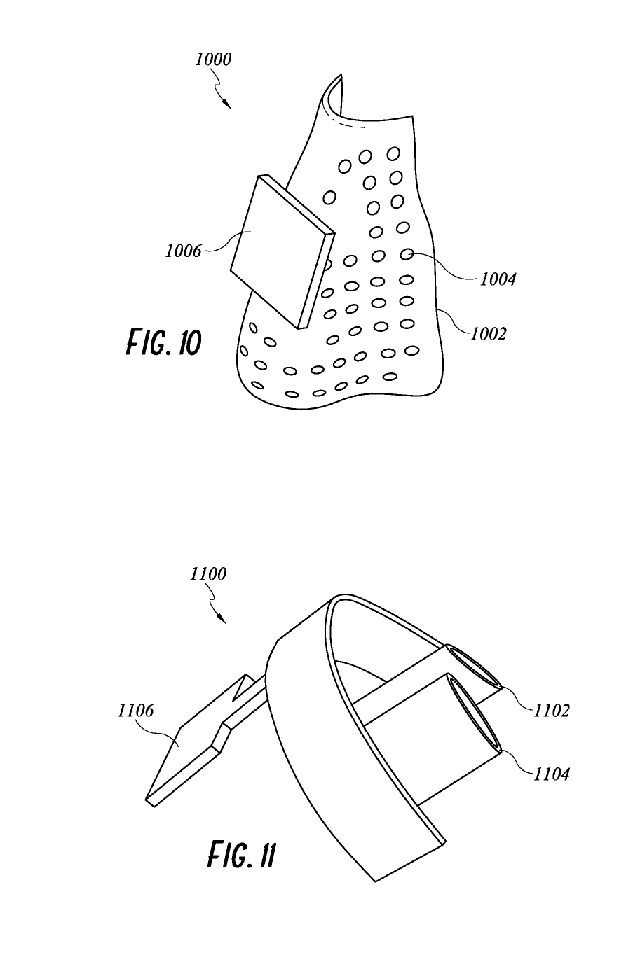

As discussed above, different shapes and/or materials can be used to form a substrate 106, one or more mounts 104, and/or one or more optical markers 102. FIG. 10 illustrates an anterior perspective view of another example optical marker apparatus. As illustrated in FIG. 10, in certain embodiments, an optical marker apparatus or device 1000 can comprise a substrate 1002 that substantially conforms to the shape of a subject's nose. The substrate 1002 can be configured to be placed over a subject's nose for attachment. The substrate 1002 can comprise one or more holes 1004 to provide adhesion and provide sufficient prevention of slippage of the optical marker apparatus 1000. The optical marker apparatus 1000 can also comprise a mount 1006, which can comprise one or more features similar to one or more mounts 104 described above.

In addition, an optical marker apparatus 100 can be adapted to attach to a different portion of a patient's body and/or face in other embodiments. FIG. 11 illustrates an anterior perspective view of another example optical marker apparatus. In the embodiment shown in FIG. 11, an optical marker apparatus 1100 can comprise one or more protruding portions 1102, 1104 that are adapted to be inserted comfortably into the nostrils of a patient. By inserting the protruding portions 1102, 1104 into nostrils of a patient, in some embodiments, an optical marker apparatus 100 can be sufficiently stable as to provide sufficiently stable tracking data from the one or more optical markers 102, which can be attached to one or more mounts 1106. The one or more mounts 1106 can comprise one or more features similar to one or more mounts 104 described above.

In yet other embodiments, an optical marker apparatus 100 can be constructed as a mouth-piece or a mouth-bite adapted to be inserted into a patient's mouth. In certain embodiments, an optical marker apparatus 100 can be configured such that a patient can bite the optical marker apparatus 100 as to maintain the position of the optical marker apparatus 100 substantially stable.

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

What is claimed is:

1. An optical marker apparatus for tracking motion of a subject during a medical imaging scan, the optical marker apparatus comprising:
a substrate configured to be attached on a nose bridge of the subject of the medical imaging scan, the substrate comprising a flexible material configured to be shaped to substantially cover a curvature of the nose bridge of the subject to at least partially contact the nose bridge of the subject, the substrate comprising a longitudinal axis;
a first mount connected to a surface of the substrate, wherein the first mount is configured to be placed over a first side of a nose of the subject, wherein the first mount comprises a rigid material;
a second mount connected to the surface of the substrate, wherein the second mount is configured to be placed over a second side of the nose of the subject, wherein the second mount comprises a rigid material, wherein the first mount and the second mount are located along the longitudinal axis of the substrate,
wherein each of the first mount and the second mount comprises a top surface, wherein the top surface is angled such that it is not parallel to the first side of the nose and the second side of the nose, wherein the top surface comprises one or more patterns, wherein the one or more patterns forms an indicator;
a first optical marker and a second optical marker, the first optical marker configured to be attached to the top surface of the first mount, and the second optical marker configured to be attached to the top surface of the second mount,
wherein at least one or more of the first optical marker and the second optical marker are configured to be identified by one or more optical detectors for tracking motion of the subject during the medical imaging scan.

2. The optical marker apparatus of claim 1, further comprising a third mount, wherein the third mount is connected to the surface of the substrate, and a third optical marker, wherein the third optical marker is configured to be attached to a top surface of the third mount.

3. The optical marker apparatus of claim 2, wherein the third optical marker is configured to be placed substantially above the nose bridge of the subject.

4. The optical marker apparatus of claim 2, wherein only a central portion of the third mount is connected to the substrate.

5. The optical marker apparatus of claim 2, wherein the third mount comprises a top surface, wherein the top surface of the third mount is substantially parallel to the substrate before attaching to the subject.

6. The optical marker apparatus of claim 1, further comprising a third mount, wherein the third mount is connected to the surface of the substrate, wherein the third mount comprises one or more pillars, wherein a third optical marker is configured to be attached to the one or more pillars.

7. The optical marker apparatus of claim 1, wherein a central portion of the substrate comprises a void space.

8. The optical marker apparatus of claim 7, wherein the void space is configured to be formed after the substrate is attached to the nose bridge of the subject by removing some or all of the central portion.

9. The optical marker apparatus of claim 1, wherein a central portion of the substrate comprises a removable portion configured to reduce a thickness of the central portion upon removal of the removable portion.

10. The optical marker apparatus of claim 1, wherein a central portion of the substrate is thinner than a terminal portion of the substrate.

11. The optical marker apparatus of claim 1, wherein the substrate is configured to prevent substantial movement of the second mount by an isolated movement of the first mount.

12. The optical marker apparatus of claim 1, wherein the top surface of the first mount is angled such that the first optical marker is orthogonal to a line of sight of the optical detector when the first optical marker is attached to the first mount.

13. The optical marker apparatus of claim 1, wherein the top surface of the first mount comprises a recessed area for affixing the first optical marker.

14. The optical marker apparatus of claim 1, wherein the top surface of the first mount comprises a recessed portion and the first optical marker comprises a protruding portion, wherein the protruding portion of the first optical marker is configured to be inserted into the recessed portion to attach the first optical marker to the first mount.

15. The optical marker apparatus of claim 1, wherein the indicator is configured to provide a visual guide to a user for attaching the first optical marker to the first mount and for attaching the second optical marker to the second mount.

16. The optical marker apparatus of claim 1, wherein the first optical marker is configured to be attached to the first mount by a chemical adhesive.

17. An optical marker apparatus for tracking motion of a subject during a medical imaging scan, the optical marker apparatus comprising:
a substrate configured to be attached on a nose bridge of the subject of the medical imaging scan, the substrate comprising a flexible material configured to be shaped to substantially cover a curvature of the nose bridge of the subject to at least partially contact the nose bridge of the subject, the substrate comprising a longitudinal axis;
a first mount connected to a surface of the substrate, wherein the first mount is located at a terminal end of the substrate, wherein the first mount comprises a rigid material;
a second mount connected to the surface of the substrate, wherein the second mount is located at a central portion of the substrate, wherein the second mount comprises a rigid material, wherein the first mount and the second mount are located along the longitudinal axis of the substrate,
wherein each of the first mount and the second mount comprises a top surface, wherein the top surface comprises an indicator;
a first optical marker and a second optical marker, the first optical marker configured to be attached to the top surface of the first mount, and the second optical marker configured to be attached to the top surface of the second mount, wherein the indicator provides visual guidance to a user for attaching the first mount and the second mount in a correct orientation,
wherein at least one or more of the first optical marker and the second optical marker are configured to be identified by one or more optical detectors for tracking motion of the subject during the medical imaging scan.

18. The optical marker apparatus of claim 17, wherein an isolated movement of the first marker does not cause the second marker to substantially move.

19. The optical marker apparatus of claim 17, wherein the first optical marker and the second optical marker are positioned such that at least one of the first optical marker and the second optical marker are viewable by one or more detectors.

20. The optical marker apparatus of claim 17, wherein the central portion of the substrate comprises a narrower width than the terminal end of the substrate.

21. The optical marker apparatus of claim 17, wherein one or more areas of the central portion of the substrate are removable.

22. An optical marker apparatus for tracking motion of a subject during a medical imaging scan, the optical marker apparatus comprising:
a substrate configured to be attached on a nose bridge of the subject of the medical imaging scan, the substrate comprising a flexible material configured to be shaped to substantially cover a curvature of the nose bridge of the subject to at least partially contact the nose bridge of the subject, the substrate comprising a longitudinal axis, wherein the substrate comprises:
two terminal portion along a longitudinal axis of the substrate; and
a central portion between the two terminal ends along the longitudinal axis of the substrate, wherein the central portion is configured to be more flexible than the two terminal ends;
a first mount connected to a surface of the substrate, wherein the first mount is located at one of the two terminal ends of the substrate, wherein the first mount comprises a rigid material;
a second mount connected to the surface of the substrate, wherein the second mount is located at the central portion of the substrate, wherein the second mount comprises a rigid material, wherein each of the first mount and the second mount comprises a top surface;
a first optical marker and a second optical marker, the first optical marker configured to be attached to the top surface of the first mount, and the second optical marker configured to be attached to the top surface of the second mount,
wherein at least one or more of the first optical marker and the second optical marker are configured to be identified by one or more optical detectors for tracking motion of the subject during the medical imaging scan.

23. The optical marker apparatus of claim 22, wherein the central portion of the substrate comprises a void space.

24. The optical marker apparatus of claim 23, wherein the void space is configured to be formed after the substrate is attached to the nose bridge of the subject by removing some or all of the central portion.

25. The optical marker apparatus of claim 22, wherein the central portion of the substrate comprises a removable portion configured to reduce a thickness of the central portion upon removal of the removable portion.

26. The optical marker apparatus of claim 22, wherein the central portion of the substrate is thinner than the two terminal portions of the substrate.

27. The optical marker apparatus of claim 22, wherein an isolated movement of the first marker does not cause the second marker to substantially move.
